(19) United States
(12) Patent Application Publication
(10) Pub. No.: US 2007/0026377 A1
(43) Pub. Date: Feb. 1, 2007

(54) Methods for preserving nucleated mammalian cells

(75) Inventors: John H. Crowe, Davis, CA (US); Fern Tablin, Davis, CA (US); Ann E. Oliver, Sacramento, CA (US); Kamran Jamil, Woodland, CA (US); Xiaocui Ma, Davis, CA (US); James S. Clegg, Bodega Bay, CA (US); Willem F. Wolkers, Davis, CA (US); Thurein Htoo, Jersey City, NJ (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 10/575,832

(22) PCT Filed: Oct. 18, 2004
(86) PCT No.: PCT/US04/34605

§ 371 Date: Apr. 13, 2006

Related U.S. Application Data
(63) Continuation of application No. 10/686,904, filed on Oct. 16, 2003, now abandoned.
(63) Continuation of application No. 10/722,154, filed on Nov. 25, 2003, now abandoned.
(63) Continuation of application No. 10/721,557, filed on Nov. 25, 2003.
Application No. 10/721,557 is a continuation-in-part of application No. 10/686,904, filed on Oct. 16, 2003, now abandoned.
Application No. PCT/US04/34605 is a continuation of application No. 10/721,678, filed on Nov. 25, 2003.
Application No. 10/721,678 is a continuation-in-part of application No. 10/052,162, filed on Jan. 16, 2002, now Pat. No. 6,770,478.
Application No. 10/052,162 is a continuation-in-part of application No. 09/927,760, filed on Aug. 9, 2001, now abandoned.
Application No. 10/052,162 is a continuation-in-part of application No. 09/828,627, filed on Apr. 5, 2001, now Pat. No. 6,723,497.
Application No. 10/052,162 is a continuation-in-part of application No. 09/501,773, filed on Feb. 10, 2000, now abandoned.

(51) Int. Cl. A01N 1/02; A61K 31/7012

(52) U.S. Cl. 435/2; 514/53

(57) ABSTRACT

Methods and compositions are provided for increasing the survival of nucleated mammalian cells following drying and rehydration. The methods include introducing a disaccharide such as trehalose into said cells, optionally including heat shock proteins, apoptosis inhibitors, and arbutin, drying said cells, and rehydrating them. The invention further provides nucleated mammalian cells that have increased capacity to survive, divide and, in some cases, differentiate, following drying and rehydration. The cells comprise a disaccharide and one or more of the following: a heat shock protein, an apoptosis inhibitor, and arbutin.

### STATEMENT AS TO RIGHTS TO INVENTIONS MADE UNDER FEDERALLY SPONSORED RESEARCH AND DEVELOPMENT

This invention was made with Federal support under Grant Nos. N66001-00-C-8048 and N66001-02-C-8055 awarded by the Defense Advanced Research Projects Agency and Grant No. HL57810 and HL61204 awarded by the National Institutes of Health. The Government has certain rights in the invention.

### CROSS-REFERENCES TO RELATED APPLICATIONS

This patent application claims priority from U.S. patent application Ser. No. 10/686,904, filed Oct. 16, 2003, U.S. patent application Ser. No. 10/721,557, filed Nov. 25, 2003, U.S. patent application Ser. No. 10/721,678, filed Nov. 25, 2003 and U.S. patent application Ser. No. 10/722,154, filed Nov. 25, 2003. The contents of these applications are hereby incorporated by reference.

### REFERENCE TO A “SEQUENCE LISTING,” A TABLE, OR A COMPUTER PROGRAM LISTING APPENDIX SUBMITTED ON A COMPACT DISK.

NOT APPLICABLE

### FIELD OF THE INVENTION

Embodiments of the present invention generally broadly relate to biological samples, such as mammalian (e.g., human) nucleated cells, such as stem cells, epithelial cells, and cells of the immune system. More specifically, embodiments of the present invention generally provide for the preservation and survival of such cells.

Embodiments of the present invention also generally broadly relate to the therapeutic and in vitro uses of biological samples; more particularly to manipulations or modifications of biological samples, -such as loading biological samples with solutes (e.g., carbohydrates, such as trehalose) and preparing dried compositions that can be re-hydrated at the time of application. When dried biological samples of the present invention are rehydrated, they are restored to viability.

### BACKGROUND OF THE INVENTION

Transporting and storing mammalian cells for in vitro and in vivo use has been difficult due to the need of the cells for acceptable temperatures, continued nutrients, and in some cases, reduced oxygen tension. Currently, nucleated mammalian cells are stored by freezing them in liquid nitrogen vapor, which requires introduction of a cryoprotectant, such as dimethyl sulfoxide (DMSO) into the cells, and freezing them to approximately −152° C. Besides the bulky equipment and supplies needed for such storage, this process creates other problems. At the concentrations required to serve as a cyroprotectant, DMSO is toxic to cells at physiological temperatures due to hydrophobic interactions with the proteins and membranes, and thus extensive washing of the cells is required following thawing. The thawing and washing procedures can reduce cellular viability and recovery, which could then affect clinical efficacy.

Dehydrating cells represents an alternative to current approaches to storing cells. It has been shown effective for the storage of human blood platelets at room temperature for up to 2 years, during which time recovery and response to thrombin remained essentially unchanged. Unfortunately, methods that are useful for platelets and methods that are useful for red blood cells are not useful for nucleated mammalian cells. Efforts to dry nucleated cells have also been reported, but achieving consistent results of highly viable, physiologically active cells following dehydration to low water contents remains elusive.

The dehydration and rehydration steps themselves are extremely stressful to the biological samples, thus protective compounds are required to safeguard the membranes and proteins during these procedures, analogous to the use of cryoprotectants during freeze-thaw cycles. Trehalose, a disaccharide found in high concentrations in many desiccation-tolerant animals and plants has been the excipient of choice for many cellular dehydration studies, due to its ability to replace the hydrogen bonded water molecules in the dehydrated samples, its high glass transition temperature, and the stability of the glycosidic bond. Unfortunately, mammalian cells lack a transporter for trehalose, and various methods, such as inducing pores in the cells for brief periods or transfecting cells, have been tried in attempts to load mammalian cells with trehalose in amounts sufficient to provide protection during drying and rehydration.

It would be desirable to have improved methods for dehydrating and rehydrating nucleated cells. The present invention fills these and other needs.

### BRIEF SUMMARY OF THE INVENTION

The invention provides methods and compositions for improving the viability and activity of mammalian nucleated cells that are dried and rehydrated.

In a first group of embodiments, the invention provides methods for loading a disaccharide into mammalian nucleated cells, comprising: contacting said cells for at least 2 hours with a solution comprising a disaccharide, thereby loading the cells with disaccharide to produce disaccharide-loaded mammalian nucleated cells. In some embodiments, the cells are stem cells, immune system cells, or epithelial cells. The contacting can be for 10 hours, or 24 hours. The disaccharide can be, for example, sucrose, maltose or trehalose, but is preferably trehalose. The solution can further comprise not more than 3% dimethyl sulfoxide.

In another group of embodiments, the invention provides methods for increasing survival of mammalian nucleated cells following drying and rehydration, comprising: (a) contacting the cells with a solution comprising a disaccharide for at least 2 hours, thereby producing disaccharide-loaded cells, (b) drying the disaccharide-loaded cells to a residual water content between 0.2 and 0.5 gram water per gram of dry weight, and (c) rehydrating the cells, thereby increasing survival of the cells. The contacting may be for 24 hours. The cells may be, for example, stem cells, immune system cells, or epithelial cells. The disaccharide can be, for example, sucrose, maltose or trehalose, but is preferably trehalose. The cells may further comprise a heat shock protein. The heat shock protein may be induced by exposing said cells to a heat shock. The heat shock may consist of raising the temperature of medium contacting the cells to 42-44° C. for one hour, and then allowing the temperature of the medium to drop to 36-38° C. Alternatively, the heat shock protein may be introduced into the cells by contacting the cells with a solution comprising the protein. Further, the heat shock protein may be expressed from a nucleic acid sequence introduced into the cells. The heat shock protein may be p26 from Artemia franciscana. The cells may be contacted with a solution comprising an apoptosis inhibitor. The apoptosis inhibitor may be selected from the group consisting of N-(2-Quinolyl)valyl-aspartyl-(2,6-difluorophenoxy)methyl ketone (in which the aspartyl residue is o-methylated or non-o-methylated), caspase I inhibitor II, calpain inhibitor, and Bcl-xL. Further, the cells may be contacted by a solution comprising arbutin or hydroquinone, provided that said cells are not 293 cells or B cells. The cells may also be contacted by a solution comprising not more than 3% dimethyl sulfoxide. In some embodiments, the cells are contacted by a solution comprising both a heat shock protein and an apoptosis inhibitor. The solution may further comprise not more than 3% dimethyl sulfoxide. Cells contacted with a solution comprising arbutin or hydroquinone are preferably dried in a medium comprising arbutin or hydroquinone. The cells are preferably dried in rounded droplets of drying buffer.

In yet a further set of embodiments, the invention provides methods for increasing survival of mammalian nucleated cells following drying and rehydration, comprising: (a) contacting the cells with a solution comprising an apoptosis inhibitor, thereby loading the cells with the apoptosis inhibitor, to produce apoptosis inhibitor-loaded cells, (b) drying said apoptosis inhibitor-loaded cells, and (c) rehydrating the cells, thereby increasing survival of the cells. The apoptosis inhibitor may be, for example, N-(2-Quinolyl)valyl-aspartyl-(2,6-difluorophenoxy)methyl ketone (in which the aspartyl residue is o-methylated or non-o-methylated), Caspase I inhibitor II, Calpain inhibitor, and Bcl-xL. The cells may be, for example, stem cells, immune system cells, and epithelial cells The cells are preferably dried in droplets of drying buffer.

In yet a further set of embodiments, the invention provides methods for increasing survival of mammalian nucleated cells following drying and rehydration, comprising: (a) introducing a heat shock protein into, or inducing production of a heat shock protein in, said cells, to produce heat shock protein-loaded cells, (b) drying said heat shock protein-loaded cells, and (c) rehydrating the cells, therebly increasing survival of the cells. The heat shock protein may be p26 from Artemia franciscana. The heat shock protein may be introduced into the cells by incubating the cells in a medium comprising the heat shock protein. The heat shock protein may be induced in said cells by raising the temperature of medium contacting the cells to 42-44° C. for one hour, and then allowing the temperature of the medium to lower to 36-38° C. The heat shock protein may be introduced into the cells by introducing into the cells a nucleic acid sequence comprising a promoter operably linked to a sequence encoding the heat shock protein. The cells can be, for example, stem cells, immune system cells, or epithelial cells. The cells are preferably dried in droplets of drying buffer.

In yet a further set of embodiments, the invention provides methods for increasing survival of mammalian nucleated cells following drying and rehydration, provided said cells are not 293 cells or B cells, comprising: (a) incubating said cells with a compound selected from arbutin and hydroquinone, to produce arbutin- or hydroquinone-loaded cells, (b) drying the arbutin- or hydroquinone-loaded cells, and (c) rehydrating said cells, thereby increasing survival of the cells. In some embodiments, the compound of step (a) is arbutin.

In yet a further set of embodiments, the invention provides isolated mammalian nucleated cells comprising a disaccharide and a compound selected from the group consisting of arbutin and hydroquinone. In some embodiments, the compound is arbutin. In some embodiments, the cell is dried. In some embodiments, the cell further comprises an apoptosis inhibitor. In some embodiments, the cell further comprises a heat shock protein. The disaccharide can be, for example, sucrose, maltose or trehalose or a mixture of these, but is preferably trehalose.

In yet a further set of embodiments, the invention provides isolated dried mammalian nucleated cells comprising a disaccharide and an exogenous heat shock protein. The disaccharide can be, for example, sucrose, maltose or trehalose or a mixture of these, but is preferably trehalose.

In yet a further set of embodiments, the invention provides isolated dried mammalian nucleated cells comprising a disaccharide and an exogenous apoptosis inhibitor. The disaccharide can be, for example, sucrose, maltose or trehalose, but is preferably trehalose.

### BRIEF DESCRIPTION OF THE DRAWINGS

FIG. 1 is a graph of viability (%) subsequent to drying vs. water content (gm. water/gm. dry weight) after drying for transfected 293 cells (T-293 cells) and for control 293 cells (293-cells), with both the transfected 293 cells and the control 293 cells having no trehalose internally and with the drying buffer for both transfected 293 cells and control 293 cells having no trehalose.

FIG. 2 is a graph of viability (%) subsequent to drying vs. water content (gm. water/gm. dry weight) after drying for transfected 293 cells (T-293 cells) and for control 293 cells (293-cells), with the transfected 293 cells and the control 293 cells both having no trehalose internally and with the drying buffer for both transfected 293 cells and control 293 cells having 150 mM trehalose.

FIG. 3 is a graph of viability (%) subsequent to drying vs. water content (gm. water/gm. dry weight) after drying for transfected 293 cells (T293 cells) and for control 293 cells (293-cells), with both the transfected 293 cells and control 293 cells having trehalose internally and with the drying buffer for both the transfected 293 cells and control 293 cells having 150 mM trehalose.

FIG. 4 is a graph of the number of colonies formed after rehydration vs water content after drying the transfected 293 cells (T293 cells) and the control 293 cells (293-cells) to 0.3 gm. water/gm. dry weight and to 0.2 gm. water/gm. dry weight, with both the transfected 293 cells and the control 293 cells having trehalose internally and with both having about 150 mM trehalose in the drying buffer.

FIG. 5 is a graph of survival (% viability) vs. water content (gm. water/gm. dry weight) for a first batch of p26-transfected 293 cells (T293 cells) after air drying and rehydration, and for a second batch of p26-transfected 293 cells (T293 cells) after vacuum drying and rehydration, with both batches of the transfected 293 cells having trehalose internally and with the drying buffer for both batches containing 150 mM trehalose. Both batches were loaded with trehalose for 24 hours by incubation at 37 C with 100 mM trehalose. The cells were dried by either air-drying or by vacuum drying and the viability after rehydration was determined by trypan blue exclusion. Air drying was conducted by at room temperature in a modified desiccator flushed with dry air at approximately 200 mL/min. Vacuum dried samples were placed in a vacuum chamber at room temperature and subjected to a vacuum of approximately 3 inches Hg. The vacuum dried samples show a significantly left-shifted curve compared to the air-dried samples, indicating much higher viability at lower water levels.

FIG. 6 is a graph of survival (% viability) vs. water content (gm. water/gm. dry weight) for a first batch of transfected 293 cells (T293 cells) after air drying while in a thin film configuration and after rehydration, and for a second batch of transfected 293 cells (T-293 cells) after air drying while in a plurality of droplets configuration and after rehydration, with both batches of the transfected 293 cells having trehalose internally and with the drying buffer for both batches containing 150 mM trehalose.

FIG. 7 is a graph of survival (% viability) vs. water content (gm. water/gm. dry weight) for a first batch of transfected 293 cells (T293 cells) after vacuum drying while in a thin film configuration and after rehydration, and for a second batch of transfected 293 cells (293 cells) after vacuum drying while in a plurality of droplets configuration and after rehydration, with both batches of the transfected 293 cells having trehalose internally and with the drying buffer for both batches containing 150 mM trehalose.

FIG. 8 is a graph of survival (% average viability) vs. water content (gm. water/gm. dry weight) for 293 cells (293 cells) when vacuum dried in 50 μL droplets and after rehydration, and for transfected 293 cells (T293 cells) when vacuum dried in 50 μL and after rehydration, with both the 293 cells and the transfected 293 cells having trehalose internally and with the drying buffer for both batches containing 150 mM trehalose. Although both types show improved viability with this combination compared to air-dried samples, the transfected cells showed higher survival than standard cells at water contents below 2 g H₂O/g dry weight.

FIGS. 9A and 9B are a flow chart showing preferred embodiments for performing the methods of the invention using human cells.

### DETAILED DESCRIPTION

I. Introduction

As noted in the Background, human nucleated cells lack a transporter for trehalose, and a number of approaches have been tried to load such cells with amounts of trehalose that will protect the cells during drying and subsequent rehydration. Surprisingly, we have now discovered that cells can be loaded with protective amounts of trehalose by endocytosis, provided that they incubated in a medium containing trehalose for a sufficient time. We have further found that addition of certain other agents to the medium (or, in the case of one group of agents, the induction of the agents or transfection of the cells with the agents) result in yet further improvements in the percentage of the cells that are viable after rehydration or in their ability to divide and, where appropriate, differentiate. If desired, one or more of the other agents can be combined in the cells to increase their survival following drying and rehydration.

The methods provided herein are generally applicable to nucleated mammalian cells, such as canine, feline, bovine, and equine cells, more preferably primate cells, and even more preferably, human nucleated cells. The methods of the invention provide the ability to dry such cells to permit them to be transported and stored, and later rehydrated. The methods of the invention can be used to dry widely differing cell types, such as (a) stem cells, including mesenchymal stem cells (“MSCs”), embryonic stem cells (“ESCs”) and cord blood stem cells (“CBSCs”), and cells that are partially differentiated from these cells, but which still retain the ability to further differentiate into terminally differentiated cells, (b) immune system cells, such as B cells, and (c) epithelial cells. Following drying by the methods of the invention, the cells can be rehydrated and restored to viability.

Because living cells must generally be maintained under physiologically acceptable conditions (for example, conditions of temperature, ambient gas content and relative humidity suitable for tissue culture), cells must generally be transported quickly. This requires the use of courier services and makes transport of cells over extended distances expensive and logistically complex. The alternative to date has been to freeze the cells in liquid nitrogen. Cells frozen in liquid nitrogen must be shipped in containers which can safely hold liquid nitrogen or dry ice, which are bulky and which creates logistical difficulties. The ability to dry mammalian nucleated cells for even a few days, to be able to ship them under simple refrigeration, and to restore them to viability therefore reduces the cost and difficulty of distributing the cells.

II. Methods of the Invention

A. Disaccharide Loading

Trehalose is known to stabilize cell membranes and proteins during dehydration. The cells of humans and other mammals, however, do not have a transporter for trehalose. Accordingly, efforts to load trehalose into mammalian cells have typically employed methods to overcome this problem, such as by creating pores in the cells to allow entry of trehalose or transfecting cells in an attempt to have them produce their own trehalose. Such methods have typically involved brief exposure of the cells to the trehalose-containing medium, typically for ten to fifteen minutes and usually for less than an hour.

In one embodiment, the present invention relates to the surprising discovery that mammalian cells incubated in a trehalose-containing medium will take up trehalose by endocytosis. While simple, this discovery has allowed us to load cells with trehalose without the more complicated procedures, such as creating pores in the cells, that the art has taught are necessary to get sufficient levels of intracellular trehalose (e.g., from 15-50 mM trehalose) to be useful in drying the cells.

The methods are therefore suitable for any nucleated mammalian cell that has sufficient endocytotic processes to take in enough trehalose from an extracellular medium during a 24 hour period to increase cell viability compared to not having trehalose present. Whether any particular nucleated mammalian cell has sufficient endocytotic processes to take in adequate amounts of trehalose from an extracellular medium can be determined by routine assays, such as the lucifer yellow assay described herein. Use of the term “biological samples” below refers to nucleated mammalian cells. Similarly, the word “cells” as used herein, unless otherwise indicated, refers to nucleated mammalian cells.

First, the cells of interest are incubated in a standard growth medium containing trehalose to load the cells with trehalose (this medium will hereafter be referred to as the “incubation medium” or the “loading buffer”). Optionally, prior to the incubation, the cells may be cultured in a standard growth medium to increase the cell population. We have found that incubation should be at least 10 hours, to give the cells enough time to take up enough trehalose to be protective, but not much over 48 hours, as the amount of trehalose taken into the cell plateaus, and cell viability begins to drop after 48 hours of incubation, as can be determined by using a standard trypan blue viability assay. Therefore, the incubation is preferably from 10 to 48 hours, more preferably from 15 to 40 hours, and yet more preferably from about 20 hours to about 30 hours. An incubation of about 24 hours is preferred.

If desired, the incubation period for loading can be shortened by “stressing” the cells by, for example, omitting glucose or fetal bovine serum from the incubation medium. This increases the rate of endocytosis, and accordingly shortens the time needed for the trehalose or other disaccharide to be taken up. In these embodiments, the cells are stressed and the incubation is for 2, 3, 4, 5, 6, 7, 8, 9, 10, 11, 12, 13, 14, 15, 16, 17, 18, 19, or 20 hours. Incubations of 3 to 20 hours are generally preferred, with incubations of about 4 to 15 hours being more preferred and 10 to 12 hours being more preferred.

The incubation is typically performed at 30 to 39° C., but is preferably at the temperature considered normal for the mammal from which the cells being incubated originated. For human cells, a temperature of 37° C. is preferred. Preferably, the cells are incubated under tissue culture conditions. Tissue culture conditions for maintaining cells of vanous types have been studied with some care and are known in the art. For human cells, we prefer to incubate the cells in 5% CO₂ at 90% relative humidity. Detailed information about tissue culture conditions for animal cells, as well as media suitable for such cultures, can be found in a number of sources, such as R. Freshney, Culture of Animal Cells, Wiley-Liss, New York (3rd Ed. 1994).

Trehalose has traditionally been the most preferred disaccharide for protecting cells, in part because of its high glass transition temperature, and in part because its glycosidic bond is resistant to degradation in the endosome. The methods of the invention, however, do not require the disaccharide to have a high glass transition temperature, and we have found that sucrose, for example, can survive the endosome. Accordingly, while trehalose is particularly preferred, it is believed that other disaccharides, such as sucrose and maltose, can be used in place of trehalose in the methods of the invention.

The disaccharide should be present in the growth medium at between 50 to 200 mM. We have found that about 80 to 150 mM is satisfactory in loading trehalose into cells of various tissues (e.g., MSCs and epithelial cells), with 90 to 120 mM being better and about 100 mM being preferred. Cells that circulate in the blood, such as B cells, load better at somewhat lower concentrations of trehalose, with 50 to 100 mM being satisfactory, 60 to 85 mM being better, and about 75 mM being preferred (in the context of loading trehalose or other disaccharides, “about” means 5 mM plus or minus).

B. Use of DMSO to Improve Cellular Distribution of Trehalose

We have found that MSCs and epithelial cells load trehalose evenly. Different cell types however, may vary in their ability to distribute trehalose evenly, due perhaps to such factors as having a higher level of membrane-bound proteins. The ability of a cell type to distribute trehalose can be estimated by such techniques as by the lucifer yellow assay discussed in the Examples. Lucifer yellow (more usually “Lucifer yellow CH,” or “LYCH,” in recognition of a carbohydrazide moiety that allow the molecule to be fixable with an aldehyde fixation agent) is a commercially available (from, e.g., Biotium, Inc. Hayward, Calif.) fluorescent dye which is of similar molecular weight and polarity to trehalose. Accordingly, it is assumed that LYCH provides an approximation of how trehalose distributes within a cell type. The LYCH can be assayed by fluorescence spectroscopy to determine overall uptake. Fluorescence microscopy can also be used to determine distribution. That is, if the LYCH is still contained within the endocytotic vesicles, the staining appears punctate, but if the dye is distributed evenly, the staining appears diffuse and homogeneous. If distribution into specific subcellular compartments (e.g. mitochondria) is of concern, that can be determined by cell fractionation.

We have found that DMSO is not necessary for drying and rehydration of cells, but it does improve intracellular distribution of trehalose. It is believed that the trehalose stabilizes proteins and membranes during the drying process. Therefore, it is believed that any portions of the cell in which trehalose is absent would be more likely to sustain damage during drying and rehydration, possibly comprising viability of the cell. If the cells are not distributing trehalose evenly, therefore dimethyl sulfoxide (“DMSO”) may be added. The DMSO may be added as little as 20 minutes before the end of the contemplated trehalose loading time (that is, the time the cells are incubating in a trehalose-containing solution to load them with trehalose), more preferably 30, 40, 50 or 60 minutes before the end of the trehalose loading time. The DMSO may be added 2, 3, 4, 5, or 6 hours before the end of the trehalose-loading time. The DMSO may be added in amounts so that the DMSO constitutes between 0.1% and about 2% DMSO by volume.

The lower limit of DMSO that is suitable can be determined by introducing an amount (for example, 0.5% of DMSO) into the media, incubating for 2 hours, performing the lucifer yellow assay, and visually observing if the dye is evenly distributed. If the dye is not evenly distributed, then the test is run on a parallel group of cells with a higher percentage of DMSO to see if that provides adequate distribution.

Amounts higher than 3% are not desirable since DMSO itself becomes toxic to cells at higher concentrations. Thus, providing the DMSO at percentages of about 2% or below is preferred. Whether any particular amount between 2% and 3% is too toxic for the cells can be readily determined by introducing the amount into the media, incubating cells in the media for 2 hours, and determining the percentage of viable cells by standard cell viability assays, such as by taking a sample of the cells, diluting the sample with trypan blue and counting the viable (unstained cells) and non-viable (stained cells) on a hemocytometer under a microscope. The number of viable cells counted, divided by the total number of cells, times 100, provides the percentage of viable cells. Percentages of DMSO which result in viability of less than 50% of the cells should be avoided. More preferably, percentages of DMSO are used that result in viability of 60%, 70%, 80% of the cells or higher.

It is noted that, following the trehalose loading incubation, the cells are placed in a drying buffer, which does not contain DMSO. Thus, the buffer in which the cells are dried; and in which they are subsequently rehydrated does not contain DMSO. Further, it is believed that the amount of DMSO taken into the cells given the modest amounts added to the incubation buffer is small. Thus, the methods of the invention avoid the toxicity problems which have been associated with DMSO's use as a cryoprotective agent.

C. Use of Arbutin

Surprisingly, we have found that the ability of some cell types to survive being dried and then rehydrated is significantly enhanced by including the compound arbutin in the incubation medium. Arbutin (CAS Number 497-76-7, Beilstein Registry Number 89673), is also known as hydroquinone-beta-D-glucopyranoside, 4-hydroxyphenyl-beta-D-pyranoside, p-arbutin, and arbutine. It has the molecular formula C₁₂H₁₆O₇, and a molecular weight of 272.25. Arbutin was originally extracted from the leaves of plants such as Arctostaphylos uva-ursi (“bearberry”), and the “resurrection plant” Myrothamnus flabellifolia; natural and synthetic arbutin are now commercially available from a number of sources, including Sigma-Aldrich (St. Louis, Mo.); Kraeber GmbH & Co. (Ellerbek Germany); Thinker Chemical Co., Ltd., (Hangzhou, China), Peakchem division, City Pride Co., Ltd. (Hangzhou, China), and Shanghai UCHEM Co., Ltd. (Shanghai, China). Arbutin is used commercially in topical cosmetic agents for whitening skin.

As reported in the Examples, below, arbutin was found to enhance the metabolism of MSCs after drying and rehydration, and to enhance the ability of MSCs to divide and differentiate after drying and rehydration. Interestingly, arbutin also induced the expression of heat shock protein 70 (HSP 70) in MSCs. The biological effects of arbutin went beyond those expected from the induction of the HSP alone. Therefore, while the induction of HSP70 may be responsible for some of the effects observed, it is believed that the beneficial effect of the arbutin on cell metabolism and division is due to factors other than or in addition to the induction of the HSP alone.

As noted, arbutin is particularly useful in connection with stem cells, such as MSC, that are dried and then rehydrated. It can be added to the incubation medium preferably during the entire incubation with trehalose, but can be added part way through the incubation if desired. It is desirable that it be present for at least 15 minutes, more preferably, 30 minutes, still more preferably, 1, 2, 3, 4, 5, 6, 7, 8, 9, 10 or more hours, with longer times being more preferred to permit the cells to take in the arbutin. It can be present at 5 to 150 mM, with 10 to 100 mM being more preferred, 20 to 80 mM being still more preferred and 30 to 60 mM more preferable. We have found 40 mM to be satisfactory for loading stem cells, and use that as our preferred amount.

When arbutin is present in the incubation medium, the amount of trehalose in the medium can be reduced. In the absence of arbutin, 100 mM of trehalose is the preferred concentration in the incubation medium for loading stem cells. With arbutin present in the medium, the concentration of trehalose can be reduced to 70 mM for stem cells.

Based on our studies, however, we believe the use of arbutin will improve the viability of many cell types. It is, however, toxic to 393 and B cells and therefore if used in loading arbutin-sensitive epithelial cells or B cells, it should not be present at more than about 10 mM. Whether arbutin is toxic or beneficial to preserving cells of any particular cell type can be readily determined by standard assays, such as the trypan blue viability assay described above or the propidium iodide (PI) exclusion assay described in the Examples.

As may be apparent from the chemical names for arbutin, it is a glycosylated hydroquinone. Thus, while arbutin in particularly preferred in the methods and compositions of the invention, it is believed that hydroquinone can be substituted for arbutin in the methods and compositions described herein.

D. Heat Shock Proteins

We have further surprisingly found that the presence of heat shock proteins (“HSPs”, also known as “stress proteins”) in the cells prior to drying them enhances the viability of the cells upon rehydration. The proteins can be endogenously produced in the cells in response to heat shock, can be exogenously provided, or can be expressed as a result of transfecting the cells with a nucleic acid sequence encoding an HSP of choice.

As used herein, a “stress protein,” also known as a “heat shock protein” or “HSP,” is a protein that is encoded by a stress gene, and is therefore typically produced in significantly greater amounts upon the contact or exposure of the stressor to the organism. A “stress gene,” also known as “heat shock gene” is used herein as a gene that is activated or otherwise detectably upregulated due to the contact or exposure of an organism (containing the gene) to a stressor, such as heat shock, hypoxia, glucose deprivation, heavy metal salts, inhibitors of energy metabolism and electron transport, and protein denaturants, or to certain benzoquinone ansamycins. Nover, L., Heat Shock Response, CRC Press, Inc., Boca Raton, Fla. (1991). “Stress gene” also includes homologous genes within known stress gene families, such as certain genes within the HSP70 and HSP90 stress gene families, even though such homologous genes are not themselves induced by a stressor. Each of the terms stress gene and stress protein as used in the present specification may be inclusive of the other, unless the context indicates otherwise.

In preferred embodiments, the cells are briefly heated to a temperature that induces expression of heat shock proteins (that is, the cells are “heat shocked”). Heat shocking has been conducted on cells of many species to study the effect of HSPs and patterns of HSP expression. Accordingly, the temperatures at which to shock cells of many mammalian species can be found in the literature or readily determined following art-recognized techniques. For human cells, raising the cells (or the medium in which the cells are bathing) to a temperature of about 42-44° C. is preferred. At temperatures over 44° C., the viability of the cells begins to drop. The cells can be exposed to a quick pulse of heat, or the temperature of the medium can be gradually stepped up. A quick pulse consists of heating the medium containing the cells to the desired temperature for 20 minutes to 2 hours, with 1 hour being preferred. For non-human cells from animals with normal body temperatures higher than that of humans, correspondingly higher temperatures are useful to heat shock the cells. It can readily be determined whether any particular temperature is too hot by performing a trypan blue assay as described above. Death of more than 20% of the cells indicates that too high a temperature has been used.

Following the heat shock, the temperature of the cells (or, more precisely, of the medium comprising the cells) is allowed to drop, usually back to the same temperature as that at which the cells were being incubated or grown prior to the shock, and permitted 10-48 hours, more preferably 20-28 hours, most preferably 24 hours, to express heat shock proteins induced by the heat shock. If desired, trehalose can be introduced into the medium before the heat shock, during the heat shock, or following the heat shock during the period the cells are expressing the heat shock proteins, to permit the cells to become loaded with trehalose at the same time the heat shock proteins are being expressed, to reduce the overall time of the procedure, or trehalose can be introduced into the medium after the heat shock protein-induction period to load the cells with trehalose at that point.

The induction of HSPs can be confirmed by incubating the cells for 10-48 hours to give them time to express the induced HSPs, lysing the cells, running the cell contents on a gel to separate the proteins, transferring the proteins from the gel to a blot (e.g,, nitrocellulose or PVDF membrane) and probing the membrane with antibodies to HSP proteins. All of these procedures for determining the presence of proteins are well known in the art. Antibodies to numerous HSP proteins are commercially available. For example, Upstate Biotechnology, Lake Placid, N.Y., sells antibodies to HSP 27 and 90, while antibodies to HSP 25, 27, 56, 70, 73, 84, 86, and 104 are available from Abcam Ltd. (Cambridge, UK). (Heat shock proteins are commonly referred to by their molecular weight expressed in kiloDaltons, or “kDa”). In studies underlying the present invention, heat shocking of murine B cells resulted in the induction of HSP70.

As an alternative to inducing HSP expression in the cells, they can be incubated in a medium containing one or more HSPs of choice. It is anticipated that the cells will endocytose some amount of the HSPs during the incubation. If it appears that the HSP is not being endocytosed into the cells or that the HSP is entering the cells more slowly than desired by the practitioner, the amount of uptake can be increased by use of a protein delivery reagent, such as the BioPORTER® Quikease™ system described infra.

The cells can also be transfected with a vector encoding a heat shock protein. Numerous HSPs have been studied since at least the 1980's and their amino acid and nucleic acid sequences are known. See, e.g., Hunt, C. and Marimoto, R. Proc. Natl. Acad. Sci. USA 82:6455-6459 (1985); Drabent, B. et al., Nucleic Acids Res. 14(22): 8933-8948 (1986);

Uoshima, K., et al., Biochem. Biophys. Res. Commun. 197(3):1388-1395 (1993) (rat HSP27); Carper et al., Nucleic Acids Res. 18 (21): 6457 (1990) (human HSP27) GenBank accession number NM 212504 (rat HSP70). The studies set forth in the Examples show the results of transfecting mammalian cells with a nucleic acid encoding a brine shrimp HSP, p26, as an exemplar HSP. Based on the results seen with using p26, we believe that any of the heat shock proteins of roughly 104 kDa or smaller will work to increase viability of cells undergoing drying and rehydration. As noted, p26 is a brine shrimp protein, and protects human cells during drying and rehydration, as shown in the Examples. Accordingly, the HSP protein does not have to be a human HSP, or even a mammalian HSP.

Persons of skill will be aware that some HSPs are constitutively expressed, while others are induced when the cells are exposed to stress conditions or have their expression markedly increased under stress conditions. For purposes of the methods of the invention, HSPs that are induced under stress conditions or which have their expression increased when the cells are placed under stress are preferred, and HSPs that are induced when cells are placed under stress conditions are particularly preferred. As shown in the Examples, the HSP referred to as p26 protects human cells and is preferred for uses in which transfected cells are suitable.

It will be appreciated that numerous vectors and promoters are known in the art. The choice of the particular vector or promoter is not critical to the invention. Since it is desirable that the HSP chosen be expressed in the cells to be dried, the promoter of course should be one that will “drive” expression of the protein under the conditions of the culture. Thus, it is preferred if the coding sequence for the protein be placed under the control of a promoter that will either be constitutively active or that will be active under the culture. In a preferred embodiment, the promoter is the human cytomegalovirus immediate-early promoter/enhancer. The CMV promoter/enhancer permits efficient, high-level expression of the recombinant protein in transfected cells. Expression of recombinant HSPs are known in the art. See, e.g., Li et al., Infect Immun. 69 (5): 2878-2887 (2001). Vectors and methods for transfecting cells with HSPs, by themselves or in conjunction with other proteins, are taught, for example, in U.S. Pat. No. 6,495,347.

Although transfected cells can be used in clinical applications, induction of endogenous HSPs is preferred, since it is more difficult to get regulatory approval to introduce into patients cells that have been recombinantly engineered. Thus, for example, HSPs for cells contemplated for therapeutic applications, such as MSC, are preferably induced by heat shock or are loaded into the cells from the medium. For in vitro use, the HSPs can be endogenous or can be introduced by transfection.

In the studies reported in the Examples, human 293 epithelial cells were transfected with p26. p26 was seen to provide a protective effect on cell survival and recovery upon rehydration after drying. Further, p26 acted to inhibit apoptosis of the cells during drying. HSPs have also been found to improve viability following drying and rehydration of HeLa cells, and to decrease the presence of damaging reactive oxygen species (ROS) in HeLa cells during drying. HeLa cells are a cell line originally derived from a cervical cancer; cervical cancers are considered to be of epithelial origin. As noted above, arbutin is toxic to epithelial cells, and its use with epithelial cells is not preferred. Therefore, HSPs are particularly useful in connection with epithelial cells.

E. Apoptosis Inhibitors

We have further surprisingly found that introducing one or more apoptosis inhibitors into the incubation medium significantly enhances the viability of cells undergoing drying and then rehydration. Typically, the inhibitors interfere with caspases that are known to be involved in the apoptotic pathway. Four apoptosis inhibitors have been tested, and three were found to enhance survival of cells undergoing drying and rehydration:

(a) N-(2-Quinolyl)valyl-aspartyl-(2,6-difluorophenoxy)methyl ketone, (C₂₆H₂₅N₃O₆F₂). This compound is commercially available from MP Biomedicals (Irvine, Calif.) (MP Biomedicals, Calbiochem (San Diego, Calif.), Kamiya Biomedical Co. (Seattle, Wash.), and Imgenex (San Diego, Calif.) is a cell permeable, irreversible pan-caspase inhibitor; especially active against caspases 1, 3, 8, and 9. The compound is commercially sold as as “Q-VD-oPh” or, in MP Biomedicals parlance, “OPH-109”, which name is used in the Examples. The term Q-VD-OPH (or “Q-VD-oPh”) denotes that the compound has a quinoline derivative (Q), a dipeptide, valine (V, in standard single letter code) and aspartic acid (D, in standard single letter code), and a non toxic 2,6-difluorophenoxy methylketone (OPH) group. See, Caserta et al., Apoptosis 8(4): 345-352 (2003); Rebbaa, A. et al. Oncogene 22:2805 (2003); Melnikov, et al., J Clin Invest. 110: 1083-1091 (2002); Patil and Sharma, NeuroReport 15:981-984 (2004). The mechanism of action involves the formation of an irreversible thioether bond between the aspartic acid derivative in the inhibitor and the active site cysteine of the caspase with the displacement or the 2,6-difluorophenoxy leaving group. According to MP Biomedicals literature, Q-VD-OPH is effective in vitro at concentrations of 10 μM to 20 μM. For tissue culture studies 10 mM or 20 mM stock solutions are prepared in DMSO and diluted 1:1000 directly into the tissue culture medium. For in vivo use, Q-VD-OPH has been administered in 80% to 100% DMSO to assure solubility at the doses given. A dose of 20 mg/kg has been used most frequently, but doses of 120 mg/kg have been used in vivo studies. To reduce hydrophobicity, several of the suppliers mentioned above, such as Calbiochem, sell a version of Q-VD-oPh in which the aspartyl residue is not o-methylated.

(b) Caspase I inhibitor II (IL-1β Converting Enzyme (ICE) Inhibitor II, available from EMD Biosciences, Inc., San Diego, Calif.) a cell-permeable and irreversible inhibitor of caspase-1 (Ki=760 pM) and caspase-4, that inhibits Fas-mediated apoptosis and acidic sphingomyelinase activation;

(c) Calpain inhibitor (OXIS International, Inc., Portland, Oreg., see, e.g., Shinohara, K. et al., Biochem. J. 317:385 (1996)), a cell-permeable inhibitor of calpain I (Ki=190 nM), calpain II (Ki=220 nM), cathepsin B (Ki=150 nM, and cathepsin L (Ki=500 pM); and

(d) Bcl-xL (Biosource International, Camarillo, Calif.), a cell-permeable peptide that prevents apoptotic cell death by directly binding to the voltage-dependent anion channel (VDAC) and blocking its activity. Leads to the inhibition of cytochrome c release and loss of mitochondrial membrane potential (DYm).

Of these four, the inhibitor Q-VD-OPH was found to be the most effective at retaining cell viability, particularly of murine B cells, during drying and rehydration. Calpain inhibitor was tested only in conjunction with OPH-109 and it did not increase survival over the use of OPH-109 alone; its effect by itself was not tested.

Based on the results with these apoptosis inhibitors, it is expected that most if not all inhibitors of apoptosis will be beneficial in enhancing cell survival of drying and rehydration. Whether or not any particular apoptosis inhibitor is effective at increasing cell viability can be readily determined following the teachings of this disclosure, including the assays taught herein.

Preferred inhibitors are those that are cell-permeable, so that they can enter the cell from the incubating medium, although cells can also be loaded with the inhibitor by using a commercially available protein delivery reagent, such as BioPorter®. Numerous inhibitors of apoptosis are known and are commercially available. Calbiochem alone (a brand name of EMD Biosciences, Inc., San Diego, Calif.) sells some twenty inhibitors of various caspases.

It is known that some inhibitors are constitutively expressed and some are induced under stress conditions or are much more strongly expressed under stress conditions. Inhibitors that are induced under stress conditions or are much more strongly expressed under stress conditions are preferred in the methods of the invention.

The inhibitor is preferably present in the incubation medium at a concentration from 5 μM to 150 μM. More preferably, it is present from 10 μM to about 100 μM, more preferably from 15 μM to about 80 μM and still more preferably from about 20 μM to about 60 μM. We have had good results using apoptosis inhibitors at a concentration of 30 μM, which is accordingly the most preferred.

Interestingly, in the studies underlying the invention, trehalose was seen to block apoptosis in murine B cells during drying and rehydration. It does not, however, inhibit apoptosis due to heat shock or generally, and therefore its anti-apoptotic effect appears specific to dehydration. Trehalose is, of course, a disaccharide and by definition is not a heat shock protein.

F. Drying Buffer

Following incubation with trehalose to load the cells, the cells are harvested and placed in a drying buffer. If necessary to harvest the cells, they may be trypsinized to release them from a surface on which they have been incubated. The cells are then gently spun to pellet them. The supernatant is removed and replaced with a drying buffer. The drying buffer comprises trehalose or other disaccharide used to load the cells, which is preferably present in a concentration higher than that used in the incubation medium. The trehalose or other disaccharide is preferably present at from 100 to 200 mM, with 120 to 180 mM being preferred, and 140 to 170 mM being more preferred. We have found 150 mM to be satisfactory, and this concentration is our most preferred.

If arbutin or hydroquinone has been used in the incubation medium, than it is preferred that it also be present in the drying buffer. The arbutin or hydroquinone is preferably present at from 20 to 150 mM, with 40 to 120 mM being preferred, and 50 to 100 mM being more preferred. We find 70 mM of arbutin to be satisfactory, and this concentration is the most preferred.

The drying buffer preferably comprises a bulking agent to help separate the cells. Albumin is a preferred bulking agent. Human cells do not appear to be particularly sensitive to the type of albumin present; for human cells, human serum albumin or bovine serum albumin are both acceptable. We have found that cells of some species do not tolerate albumin of some other species. Accordingly, if the cells to be dried are from a non-human mammal, it is desirable to place a sample of the cells in culture medium to which the albumin to be used has been added and observe the cells to see whether they lyse. If they lyse, albumin from another source organism should be tested until one is found which does not cause lysis. Albumin from the same organism as that from which the cells originated will be compatible. Other polymers suitable for use as bulking agents are, for example, water-soluble polymers such as HES (hydroxy ethyl starch), polyvinyl alcohol, and dextran.

G. Drying the Cells

Nucleated mammalian cells normally cannot withstand being dried to bone dryness, contrary to claims made by some researchers. We find that the cells are preferably dried to 0.2 to 0.5 grams of residual water per gram of dry weight.

We have found that the cells can be dried in any of three ways. First, and most preferably, the cells can be dried by vacuum drying. In this embodiment, the cells are preferably dried in 50 μL aliquots. Rounded droplets are preferred to spreading the cells onto a surface. The Examples report significantly better viability of cells that are dried in rounded droplets of drying buffer rather than thin films. The cells are preferably dried at room temperature, with 20 to 25° C. being the preferred temperature range. The cells are dried under a vacuum of approximately 3 inches of mercury.

Second, the cells can be air dried. In this embodiment, the cells are preferably dried in 50 μL aliquots. Once again, rounded droplets of cells in drying buffer are preferred to spreading the cells onto a surface as a thin film. They can be dried at room temperature. In this embodiment, the cells are dried under a diffuse stream of dry air until they reach the desired range of dryness.

Third, the cells can be dried by freeze drying. In this embodiment, the cells are preferably dried in 50 μL aliquots, which is typically performed in a 10 drop array. Freeze drying can result in reducing the water content to levels below 0.2 grams per gram dry weight. To avoid this, the system is preferably calibrated by freeze drying parallel samples of the cells of choice in the drying buffer contemplated for use for different period of time in the lyophilizer to be used to determine the time points at which the cells will be dried to the desired residual water content.

H. Storing and Rehydrating the Cells

Once dried, the cells are preferably stored at 4° C. Preferably, the cells are stored under vacuum in an air-tight container to prevent exposing them to changes in ambient humidity.

When rehydrating the cells is desired, the cells can be placed directly into a growth medium standard for the cell type in question or the growth medium can be added directly to the container in which the cells are stored. Optionally, the cells are prehydrated prior to placing them in medium by exposing them to humid air. We have found, however, that a prehydration step is not necessary when rehydrating nucleated cells dried according to the methods herein because of the residual water content of the cells. Since prehydration adds a step without corresponding benefit, it is usually omitted.

The medium can be at room temperature or preferably from 25 to 39° C. (the temperature of the medium may be that normal for the species from which the cells originate if it is higher than 39° C.). Preferably, the conditions of gases, temperature, and humidity under which the cells are rehydrated are those under which the cells were loaded with trehalose.

III. Uses

Cells dried by the methods of the invention can be conveniently transported, preferably under refrigeration, Preferably, the transport is under vacuum in an air-tight container. Preferably, the container is also water-impermeable to reduce premature, inadvertent rehydration of the cells in the event of encountering high humidity conditions or accidental exposure to liquids during transport. The invention therefore provides a means for convenient shipping of dried cells, while reducing or eliminating the need for costly courier services and the special handling required for the liquid nitrogen-frozen cells of conventional techniques for preserving nucleated mammalian cells.

Cells dried and then rehydrated by the methods of the invention can be used for a number of applications. It is envisaged that epithelial cells and other cells can be used in place of conventional cells in biosensors to detect toxic substances in assays. It is further contemplated that stem cells, such as ESCs, CBSCs, or MSCs, dried according to the invention can be shipped to a location in need of such cells, rehydrated, and introduced into patients who can benefit from the presence of such cells.

IV. Definitions

Units, prefixes, and symbols are denoted in their Système International de Unites (SI) accepted form. Numeric ranges are inclusive of the numbers defining the range. Unless otherwise indicated, nucleic acids are written left to right in 5′ to 3′ orientation; amino acid sequences are written left to right in amino to carboxy orientation. The headings provided herein are not limitations of the various aspects or embodiments of the invention, which can be had by reference to the specification as a whole. Accordingly, the terms defined immediately below are more fully defined by reference to the specification in its entirety. Terms not defined herein have their ordinary meaning as understood by a person of skill in the art.

“Mammalian” means from a mammal. Canine, feline, equine, bovine and primate mammals are preferred with humans being particularly preferred.

“Nucleated” refers to a cell that have a nucleus. It does not refer to cells such as red blood cells, that had a nucleus, but lost the nucleus in the course of differentiation, but does refer to red blood cell precursors still in possession of a nucleus, or to blood platelets.

“293 cells” are a cell line of human embryonic kidney (“HEK”) cells.

“Cells of the immune system” refers to B cells, T cells, and dendritic cells.

“Arbutin” (CAS Number 497-76-7, Beilstein Registry Number 89673), is a compound also known as hydroquinone-beta-D-glucopyranoside, 4-hydroxyphenyl-beta-D-pyranoside, p-arbutin, and arbutine. It has the molecular formula C₁₂H₁₆O₇, and a molecular weight of 272.25.

“Exogenous,” when referring to the presence of heat shock proteins or apoptosis inhibitors in a cell of a given type, means a heat shock protein or apoptosis inhibitor not expressed by a normal cell of that type. For example, the heat shock protein p26 from Artemia franciscana is not expressed by human cells unless they have been altered by being transfected with a nucleic acid sequence encoding p26. The term is intended to distinguish heat shock proteins or apoptosis inhibitors introduced from outside the cell, or with which the cell is transfected, from heat shock protein or apoptosis inhibitors the cell might naturally express in response to environmental change or in response to signaling from other cells.

“Contacting” means bringing into physical contact.

V. Heat Shock Proteins

Heat shock proteins (HSP) are considered to be stress proteins. HSPs assist the folding of proteins, reduce stress-associated protein denaturation and aggregation, aid in renaturation, and influence the final intracellular location of mature proteins. Stress proteins are divided into groups or families, including Hsp100, Hsp90, Hsp70, Hsp60 (the chaperonins), and the small heat shock/α (alpha) crystallin proteins, sometimes referred to as α-Hsps. Small heat shock proteins including α (alpha) crystallin proteins are low molecular weight heat shock proteins, ranging in size from about 10- to 40-kDa monomer molecular mass, but oli-gomerize into particles of varying monomer numbers. The functions of chaperones differ, but their activities are interrelated and often dependent on association into macromolecular complexes, sometimes consisting of representatives from more than one group or family.

P26 belongs to the α crystallin or α-Hsps group or family. As with many other Hsps, α-Hsps or a crystallin proteins protect cells during stress by preventing aggregation of unfolded proteins and in some cases assisting in their renaturation. As indicated, p26 is a small heat shock/α crystallin protein, and has a diameter of about 15 nm, or about 520 kDa It has 28 subunits, each being about 20.7 kDa. When biological samples are nucleated cells, stress causes p26 to move into the nucleus of the nucleated cell.

P26 is found in the encysted embryos of the primitive crustacean Artemia franciscana (the North American brine shrimp). Encysted embryos of A. franciscana contain very large amount of the α-Hsp or p26, making up from about 12% to about 15% by weight of the total nonyolk protein. The remarkable stress resistance of Artemia cysts including p26 protects shrimp embryo cells during encystment, diapause, and anaerobic quiescence, and prevents the aggregation of other proteins when shrimp embryos experience stresses of various kinds; thus playing an important role in their growth and development.

For a comprehensive discussion of p26, including procedures on purifying p26 to homogeneity and measuring the concentration of p26, see Influence of trehalose on the molecular chaperone activity of p26, a small heat shock/α-crystallin protein by Viner and Clegg., Cell Stress Society International, Cell Stress & Chaperones (6(2), pp. 126-135 (2001)). For another comprehensive discussion of p26, including the cloning and sequencing a cDNA for p26, the listing of the complete sequence of p26-3-6-3 and the deduced amino acid sequence of p26, and the comparison of the deduced amino acid sequence of p26 to other small heat shock/α crystallin proteins (e.g., αA-crystallin, human αB-crystallin, human small heat shock protein 27 (H27), and a Drosophila small heat shock protein known as embryonal lethal (2) 13-1 (Dro)), see Molecular Characterization of a Small Heat Shock/alpha-Crystallin Protein in Encysted Artemia Embryos by Liang et al., J Biol Chem (272(30): 19051-19058 (1997)). See also, Liang et al., Purification, structure and in vitro molecular-chaperone activity of Artemia p26, a small heat-shock/alpha-crystallin protein, Eur. J. Biochem. 243 (1-2): 225-232 (1997). The protein sequence of p26 may be obtained from the National Center for Biological Information (NCBI) website under accession number AAB87967. The cDNA sequence coding for p26 is available from the National Center for Biological Information (NCBI) under accession number AF031367.

The Hsp70 family is a multi-gene family of chaperones but all members have four common features: highly conserved sequence, molecular mass about 70 kDa, ATPase activity and an ability to bind and release of hydrophobic segments of unfolded polypeptide chains. The protein known as Hsp 70, however, is the only member of the family that is strongly inducible by heat stress.

In an embodiment of the invention, the p26 gene (p26 cDNA) is ligated into the vector (such as pSecTag2A DNA) by use of T4 DNA ligase and then cloned in Escherichia coli DHSa. The p26-containing plasmid produced by ligation may be mixed in a tube and incubated at room temperature for a suitable period of time (e.g., 5 to 30 minutes) with an agent that enhances transfection before application to the biological sample(s) for transfection. For example, Lipofectamine 2000 (Invitrogen, Carlsbad, Calif.) is a cationic lipid-based transfection reagent. Other transfection reagents are known and may be used in place of Lipofectamine 2000 which will, however, be mentioned as an exemplar reagent herein. The vector may be placed in a culture solution, such as-serum-free DMEM (Dulbecco's Modified Eagle Medium), to produce a transfecting solution.

For transfecting, the biological sample(s) may be treated with the transfecting solution in any suitable manner, such as by immersing the cells in the transfecting solution for a suitable period of time (e.g., 10 to 50 minutes). In an embodiment of the invention, transfections of cells was accomplished by the use of 800 nanograms of the plasmid DNA mixture and 3 μd of Lipofectamine 2000 in 60 μl of serum-free DMEM for cells in each well of a six-well culture plate. It is to be understood that the cells may be transfected with the stress protein before being loaded with the solute, after being loaded with the solute, or simultaneously with the loading of the solute.

VI. Aspects of Loading

In some embodiment of the invention, the stress protein maybe loaded into the biological sample(s) (i.e., into non-transfected biological sample(s)) by any suitable means and/or method(s), such as by the employment of a protein-loading solution (e.g., a p26-loading solution). For this embodiment of the invention, the stress protein, preferably the stress protein in essentially pure form, would be mixed with a suitable protein-loading solution (e.g., a p26-loading solution), and the biological sample(s) would subsequently be disposed in the protein-loading solution for causing the transfer of the stress protein from the protein-loading solution into the biological sample(s). The protein-loading solution may be any suitable physiologically acceptable solution in an amount and under conditions effective to cause uptake or “introduction” of the stress protein from the protein-loading solution into the biological sample(s). Broadly, by way of example only, a physiologically acceptable solution comprises one or more of the following: the stress protein (e.g., p26), a salt solution (e.g., PBS), a protein (e.g., BSA), and a carbohydrate (e.g., a starch, an oligosaccharide, etc). In other embodiments of the invention, the physiologically acceptable solution comprises one or more of the following: the plasmid DNA mixture (e.g., the plasmid DNA mixture), Lipofectamine 2000, a salt solution (e.g., PBS), a protein (e.g., BSA), and a carbohydrate (e.g., a starch, an oligosaccharide, etc).

The loading temperature of the protein-loading solution for loading a stress protein into the biological sample(s) may be any suitable temperature, such as a temperature ranging from about 25 degrees C. to about 60 degrees C., more preferably from about 30 degrees C. to about 40 degrees C., more preferably yet from about 36 degrees C. to about 38 degrees C. The loading/incubating time for loading the stress protein may be any suitable time, such as a time ranging from about 10 minutes to about 48 hours, more preferably from about 30 minutes to about 34 hours, most preferably from about 45 minutes to about 24 hours.

In another embodiment of the invention the stress protein may be delivered into the biological sample(s) through the use of a protein delivery kit sold as the BioPORTER® Quikease Protein Delivery Kit 2 (Sigma-Aldrich Corp., St. Louis, Mo.). Suitable BioPORTER® protein delivery kits are sold under product Nos. BPQ24 and BPQ96. The BioPORTER® delivery kit has a BioPORTER® reagent which reacts quickly and interacts non-covalently with the stress protein (e.g., p26) for creating a protective vehicle for immediate delivery into biological sample(s). In embodiments of the invention the BioPORTER® reagent is incubated with the stress protein (e.g., p26) for a suitable period of time, such as from about 2 mins. to about 15 mins.(e.g., 5 mins.). Subsequently, the resulting incubated product having the stress protein is then incubated with the biological sample(s) for 1 to 8 hours (e.g., about 4 hours). In an embodiment of the invention, the BioPORTER® reagent-stress protein complex is taken up by fluid phase endocytosis, subsequently fusing with a membrane (e.g., an endosome membrane) of the biological sample(s) and releasing the stress protein into the cells(e.g., into the cytosol of the cells). The foregoing procedure may also be employed for loading the solute simultaneously with the loading of the stress protein.

Embodiments of the present invention will be explained by loading of the solute into the biological sample(s) after the biological sample(s) contain the stress protein. However, it is to be understood that the spirit and scope of the present invention includes loading or transfecting the biological sample(s) with the stress protein after the biological sample(s) is/are loaded with the solute, or simultaneously with the loading of the solute. Thus, embodiments of the invention are not to be restricted to any particular order with respect to loading, or transfecting, the biological sample(s) with the stress protein, and the loading of the solute into the biological sample(s). The loading, or transfecting, the biological sample(s) with the stress protein may be: (i) before the biological sample(s) is/are loaded with the solute; (ii) after the biological sample(s) has/have been loaded with the solute; or (iii) simultaneously with the loading of the solute.

After the biological sample(s) has/have been transfected with/by, or has/have been loaded with, a desired amount of the stress protein (e.g., p26), the biological sample(s) may then be loaded with a suitable solute. Broadly, the preparation of solute-loaded biological sample(s) containing the stress protein in accordance with embodiments of the invention comprises the steps of loading one or more biological sample(s) with a solute by placing one or more biological sample(s) in a solute solution having a solute concentration of sufficient magnitude for transferring the solute from the solution into the biological sample(s). For increasing the transfer or uptake of the solute from the solute solution, the solute solution temperature or incubation temperature has a temperature above about 25° C., more preferably above 30° C., such as from about 30° C. to about 40°0 C.

The solute solution for various embodiments of the present invention may be used for loading and/or drying and/or rehydration, or for any other suitable purpose. When the solute solution is employed for loading a solute into the biological sample(s), the solute solution may be any suitable physiologically acceptable solution in an amount and under conditions effective to cause uptake or “introduction” of the solute from the solute solution into the biological sample(s). A physiologically acceptable solution is a suitable solute-loading buffer, such as any of the buffers stated in the previously mentioned related patent applications, all having been incorporated herein by reference thereto. The solute solution may also be any suitable physiologically acceptable solution in an amount and under conditions effective for drying and/or rehydration. Therefore, the solute solution may be used as a drying buffer for drying loaded biological sample(s) and/or as a rehydration buffer for rehydrating biological sample(s) in reconstituting biological sample(s). Thus, any of the solute solutions for embodiments of the present invention may be used for any suitable purpose, including loading, drying, and rehydration.

For particular embodiments of the present invention, especially when the solute solution is being employed as a loading buffer, the solute solution comprises a solute (e.g., 50 mM to 150 mM trehalose) and a salt solution (e.g., such as PBS). In other particular embodiments of the invention, especially when the solute solution is being employed as a drying buffer and/or a rehydration buffer, the solute solution comprises one or more of the following: a salt solution (e.g., PBS), a protein, a solute and an acid (e.g., HEPES, or N-(2-hydroxyl ethyl) piperarine-N′-(2-ethanesulfonic acid)). However, it is to be understood that the solute solution comprising one or more of a salt solution, a protein, a solute, and an acid may be used for any other suitable purpose. An example of a growth medium would be DMEM.

The salt solution may be any suitable physiologically acceptable solution in an amount and under conditions effective to function as a carrier medium for a solvent, or for a mixture of a solvent, a protein and/or an acid. The salt solution may comprise KCl and NaCl, such as more particularly about 1 to 15 mM KCl and about 40 to 80 mM NaCl with pH 7.2. The salt solution may also comprise a phosphate buffered saline (PBS) solution comprising NaCl, Na₂HPO₄, and KH₂PO₄. A suitable PBS buffer comprises a buffer sold under the product name Dulbecco's PBS (DPBS, Gibco cat # 14190), or a buffer comprising 283 mOsm PBS buffer (NaCl, Na₂HPO₄, KH₂PO₄, pH 7.2).

The acid may be any suitable acid. Preferably, the acid comprises a sulfonic acid, such as, by way of example only, 5 to 20 mM HEPES (N-(2-hydroxyl ethyl) piperarine-N′-(2-ethanesulfonic acid)).

The carbohydrate for various embodiments of the invention is preferably trehalose. The amount of the preferred trehalose loaded inside the biological sample(s) ranges from about 10 mM to about 60 mM (e.g., up to about 50 mM), and is achieved by incubating the biological sample(s) to preserve biological properties during drying with a trehalose solution. The effective loading of trehalose is also preferably accomplished by means of using an elevated temperature of from greater than about 25° C. to less than about 40° C. more preferably from about 30° C. to less than about 40° C., most preferably about 37° C.

In an embodiment of the invention where the solute is to be loaded into the biological sample(s) simultaneously with the loading of the biological sample(s) with the stress protein, the solute solution comprises the stress protein, the solute, a salt solution (e.g., PBS) and optionally one or more of the following: a protein (e.g., BSA), and a carbohydrate (e.g., trehalose).

The loading temperature of the solute solution for this embodiment of the invention ranges from about 25 degrees C. to about 40 degrees C., more preferably from about 36 degrees C. to about 38 degrees C. The loading/incubating time for loading the stress protein may be any suitable time, such as a time ranging from about 10 hours to about 48 hours, more preferably from about 18 hours to about 36 hours, most preferably from about 22 hours to about 24 hours.

Albumin may serve as a bulking agent, but other polymers may be used with the same effect. Suitable other polymers, for example, are water-soluble polymers such as HES (hydroxy ethyl starch), polyvinyl alcohol, and dextran.

The solute-loaded, stress protein-contained biological sample(s) in the drying buffer may then be dried by the means described above, such as by vacuum drying, air drying, or freeze drying, all known in the art. Vacuum drying is the most preferred, with air drying less preferred to vacuum drying, and freeze drying least preferred.

The solute-loaded, stress protein-containing cells in the drying buffer may be vacuum dried in accordance with well known procedures. Biological sample(s) loaded with trehalose and producing p26 may be aliquotted into volumes of 50-150 μL and subjected to vacuum in the range of 3 inches Hg at room temperature for a period in the range of 2 to 4 hours. This vacuum drying technique would bring the water content in the biological samples) down to about 0.2 gm. H₂O/gm. dry weight.

The solute-loaded, stress protein-contained biological sample(s) in the drying buffer may be air dried in accordance with well known procedures. Biological samples loaded with trehalose and producing p26 may be aliquotted into volumes of 50 uL-1.0 mL and dried either in a biohood or in a desiccator modified to distribute a stream of dry air evenly across the surface of the biological sample(s). The drying may be conducted at room temperature for a period in the range of 6 to 10 hours. This air drying technique would bring the water content in the biological sample(s) down to about 0.2 g/m. H₂O/gM. dry weight.

If the solute-loaded, stress protein-contained biological sample(s) in the drying buffer are freeze-dried, the solute-loaded, stress protein-contained biological sample(s) in the drying buffer may be dried while simultaneously cooled to a temperature below about −32° C. A cooling, that is, freezing, rate is preferably between −30° C. and −1° C./min. and more preferably between about −2° C./min to −5° C./min. Drying may be continued until about 95 weight percent of water has been removed from the biological sample(s). During the initial stages of lyophilization, the pressure is preferably at about 10×10⁻⁶ torr. As the biological samples dry, the temperature can be raised to be warmer than −32° C. Based upon the bulk of the biological samples, the temperature and the pressure it can be empirically determined what the most efficient temperature values should be in order to maximize the evaporative water loss. Dried (e.g., freeze dried) biological sample compositions preferably have from 0.2 to 0.5% gram of water per gram dry weight.

The viability (e.g., the % viability) of biological sample(s) after drying may be determined by fluorescent live/dead analyses. There are several commercially available fluorescent live/dead kits. These kits work on the same principles as trypan blue; that is, dead biological sample(s) with compromised plasma membranes will take up membrane-impermeant dyes. A typical live/dead kit may contain a membrane permeant dye (e.g. syber green, SG, from Molecular Probes), which will stain all the biological sample(s), and a membrane-impermeant dye (e.g. propidium iodide, PI), which will stain only the dead biological. sample(s). The percentage of dead biological sample(s) is calculated by counting the PI-stained biological sample(s) and dividing by the SG-stained biological sample(s). The percentage of viable biological sample(s) is calculated by subtracting the % dead biological sample(s) from 100.

After drying and storage of the biological sample(s), the process of using such a dehydrated biological-sample composition comprises rehydrating the biological sample.

It has been discovered that the ability of dried biological sample(s) having a stress protein (e.g., p26) to proliferate and form colonies after rehydration is greater than the ability of dried biological sample(s) not having a stress protein to proliferate and form colonies. It has also been further discovered that the ability of dried biological sample(s) having a stress protein and a solute to proliferate and form colonies after rehydration is also greater than dried biological sample(s) not having a stress protein, or having a solute and no stress protein. The proliferation or the number of colonies formed by the biological sample(s) after drying and rehydration may be determined by any suitable procedure well known to those skilled in the art. By way of example only, after rehydration, the biological sample(s) may be plated into T-25 flasks and incubated at 37° C. for 7 days. The biological sample(s) may then be subsequently stained with either Coomassie blue or Hema 3, and the colonies in each flask may be counted to obtain the proliferation or the number of colonies formed by the biological sample(s) after drying and rehydration.

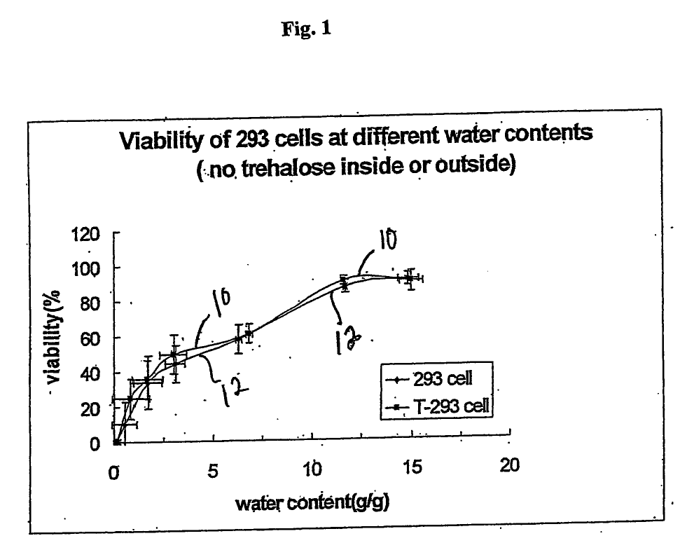

Embodiments (e.g., the viability of dried biological sample(s), proliferation of biological sample(s) after drying and rehydration, etc.) of the present invention will be illustrated with eukaryotic 293 cells (which are epithelial in origin) and by reference to FIGS. 1-10. It is to be understood that such use of 293 cells and such reference to FIGS. 1-10 are for exemplary purposes only, and are not to limit any of the embodiments of the present invention, or limit the spirit and scope of the present invention in general.

FIG. 1 shows a graph of T-293 cell viability (%) subsequent to drying vs. water content (gm. water/gm. dry weight) after drying for transfected 293 cells (T-293 cells) and for control 293 cells (293-cells), with both the transfected 293 cells and the control 293 cells having no trehalose internally and with the drying buffer for both transfected 293 cells and control 293 cells having no trehalose. Example II below provides the more specific testing conditions and parameters which produced the graphical illustrations of FIG. 1. Graph 10 and graph 12 in FIG. 1 represents the transfected 293 cells, and the control 293 cells, respectively. FIG. 1 illustrates that transfected T-293 cells transfected with p26 survive drying better than control 293 cells not having been transfected with p26. Alternatively, FIG. 1 may illustrate that, when there is no trehalose inside or outside, there is no difference in survival between the two types of cells.

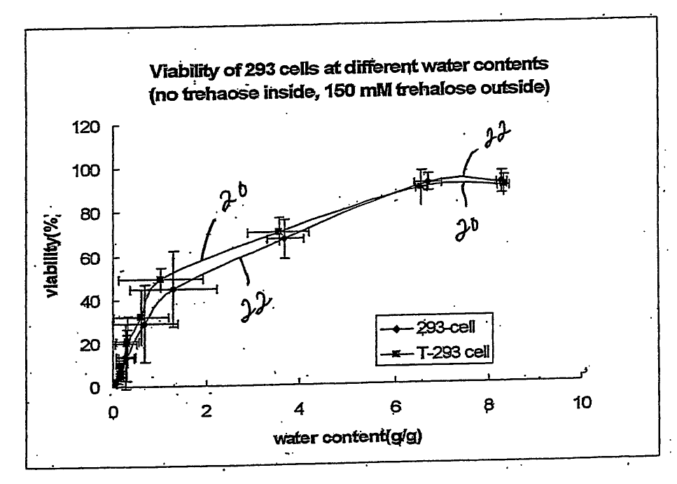

FIG. 2 is a graph of viability (%) subsequent to drying vs. water content (gm. water/gm. dry weight) after drying for transfected 293 cells (T-293 cells) and for control 293 cells (293-cells), with both the transfected 293 cells and the control 293 cells having no trehalose internally and with the drying buffer for both transfected 293 cells and control 293 cells having 150 mM trehalose. Example III below provides the more specific testing conditions and parameters which produced the graphical illustrations of FIG. 2. Graph 20 and graph 22 in FIG. 2 represents the transfected 293 cells, and the control 293 cells, respectively. FIG. 2 illustrates that survival of transfected T-293-cells is improved compared to control 293 cells when the transfected T-293 cells are dried in a drying buffer having 150 mM trehalose.

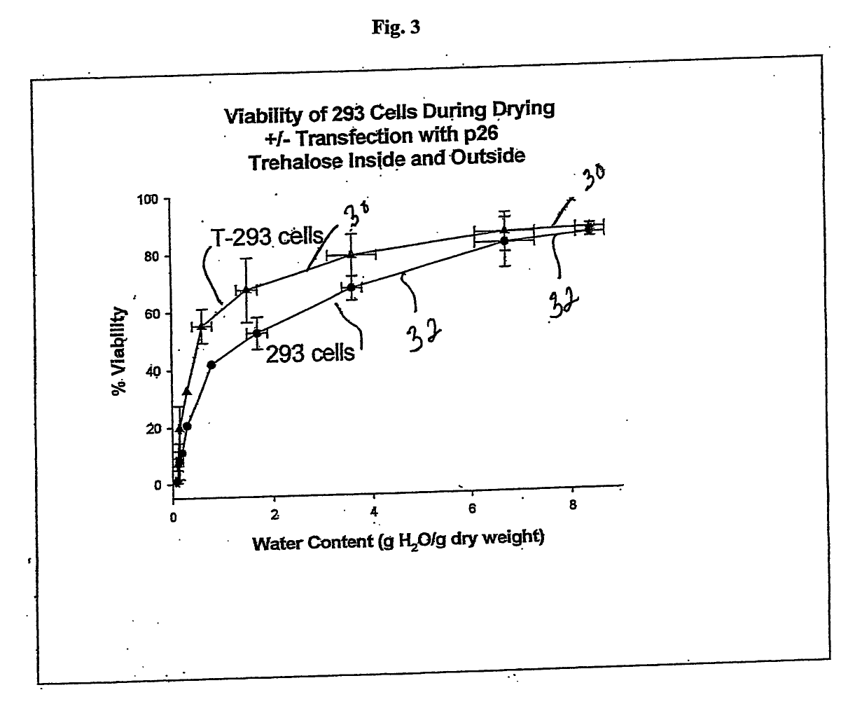

In FIG. 3 there is seen a graph of viability (%) subsequent to drying vs. water content (gm. water/gm. dry weight) after drying for transfected 293 cells (T-293 cells) and for control 293 cells (293-cells), with both the transfected 293 cells and the control 293 cells having trehalose internally and with the drying buffer for both transfected 293 cells and control 293 cells having 150 mM trehalose. Graph 30 and graph 32 in FIG. 3 represents the transfected 293 cells, and the control 293 cells, respectively. Example IV below provides the more specific testing conditions and parameters which produced the graphical illustrations of FIG. 3. FIG. 3 illustrates that mammalian T-293 cells transfected with p26, and loaded with trehalose, and dried in a drying buffer having trehalose greatly improves survival and/or viability when compared to control 293 cells not transfected with p26.

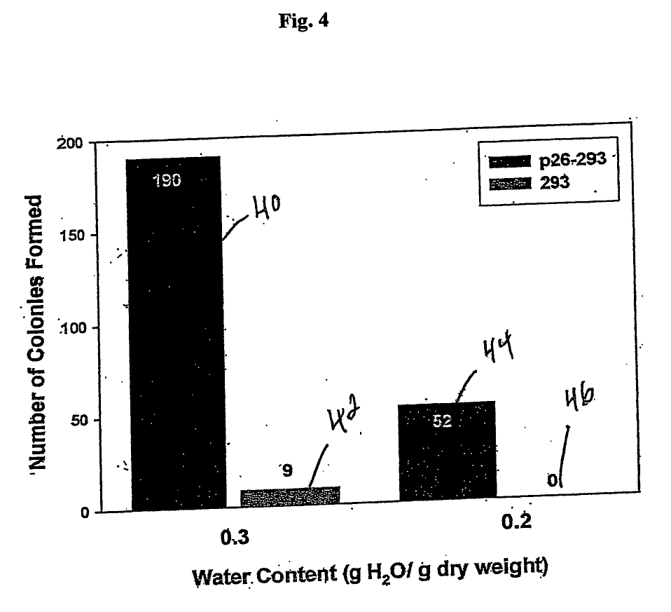

FIG. 4 is a graph of the number of colonies formed after rehydration vs water content after drying the transfected 293 cells (T-293 cells) and the control 293 cells (293-cells) to 0.3 gm. water/gm. dry weight and to 0.2 gm. water/gm. dry weight, with both the transfected 293 cells and the control 293 cells having trehalose internally and with the drying buffer for both transfected 293 cells and control 293 cells having 150 mM trehalose. Blocks 40 and 44 respectively represent transfected 293 cells for water contents of 0.3 gm water/gm dry weight and to 0.2 gm water/gm dry weight. Block 42 and the number “0” represented by numeral 46 respectively represent control 293 cells for water contents of 0.3 gm water/gm dry weight and to 0.2 gm water/gm dry weight. Example V below provides the more specific testing conditions and parameters which produced the graphical illustrations of FIG. 4. In the experiment that produced the results and graphical illustrations of FIG. 4, transfected 293 cells and control cells were both dried respectively to 0.3 gm water/gm dry weight and to 0.2 gm water/gm dry weight, rehydrated and then plated (cultured) to determine their ability to form colonies subsequent to rehydration (a measure of long-term proliferation and survival). As illustrated in FIG. 4, transfected T-293 cells dried to 0.3 gm water/gm dry weight were able to produce colonies 20× greater than the control 293 cells. This pattern persisted at lower water contents of 0.2 gm water/gm dry weight. However, no control 293 cells were able to proliferate at a water content of 0.2 gm water/gm dry weight, while a significant fraction of the transfected T-293 cells did so.

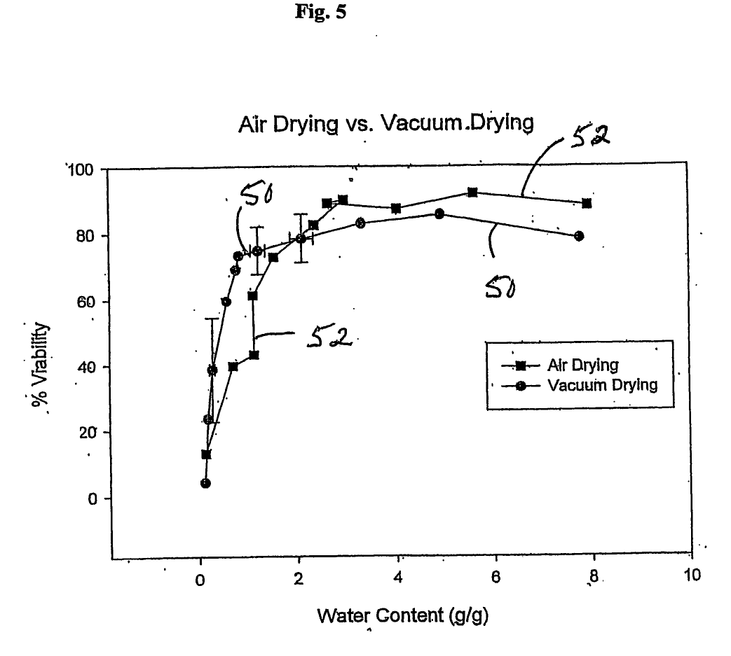

Referring in detail now to FIG. 5, there is seen a graph of survival (% viability) vs. water content (gm. water/gm. dry weight) for a first batch of p26-transfected 293 cells (T-293 cells) after air drying and rehydration, and for a second batch of p26-transfected 293 cells (T-293 cells) after vacuum drying and rehydration, with both batches of the p26-transfected 293 cells having trehalose internally. Example VI below provides the more specific testing conditions and parameters which produced the graphical illustrations of FIG. 5. Graph 50 and graph 52 in FIG. 5 represents vacuum-dried transfected 293 cells, and air-dried transfected 293 cells, respectively. FIG. 5 broadly illustrates that cell survival increases (e.g., increases by from about 20% to about 90%) by vacuum drying as opposed to air drying. FIG. 5 more specifically illustrates that after p26 transfected T-293 cells were loaded with trehalose (e.g., 25 mM to 800 mM trehalose) while incubating at a temperature above about 25° C. (e.g., from about 35° C. to about 40° C.), and then vacuum dried, instead of or as opposed to air drying, until the p26 transfected T-293 cells comprised a residual water content of less than or equal to about 2.0 grams of water per gram of dry weight of T-293 cells, survival (% viability) increases. FIG. 5 also more specifically illustrates that had the trehalose-loaded, p26 transfected T-293 cells been air dried, instead of or as opposed to vacuum dried, to the extent that the trehalose-loaded, p26 transfected T-293 cells had a residual water content of greater than (or equal to) about 2.0 grams of water per gram of dry weight of T-293 cells, survival (% viability) increases. Thus, air drying is the preferred drying technique for trehalose-loaded, p26 transfected T-293 cells if the residual water content of the trehalose-loaded, p26 transfected T-293 cells is maintained at greater than (or equal to) about 2.0 grams of water per gram of dry weight of T-293 cells (e.g., from about 2.0 grams of water per gram of dry weight of T-293 cells to about 8.0 grams water per gram of dry weight of T-293 cells); and vacuum drying is the preferred drying technique for trehalose-loaded, p26 transfected T-293 cells if the residual water content of the trehalose-loaded, p26 transfected T-293 cells is maintained at less than (or equal to) about 2.0 grams water per gram of dry weight of T-293 cells. As shown in FIG. 5, the survival of the 293 cells (i.e., the biological sample) is preferably at least about 60% (e.g., such as from about 60% to about 80%), more preferably at least about 80%.

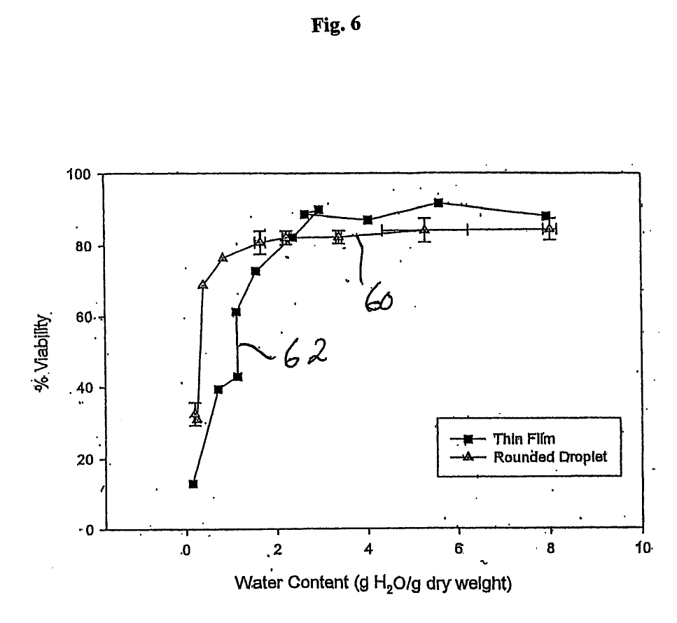

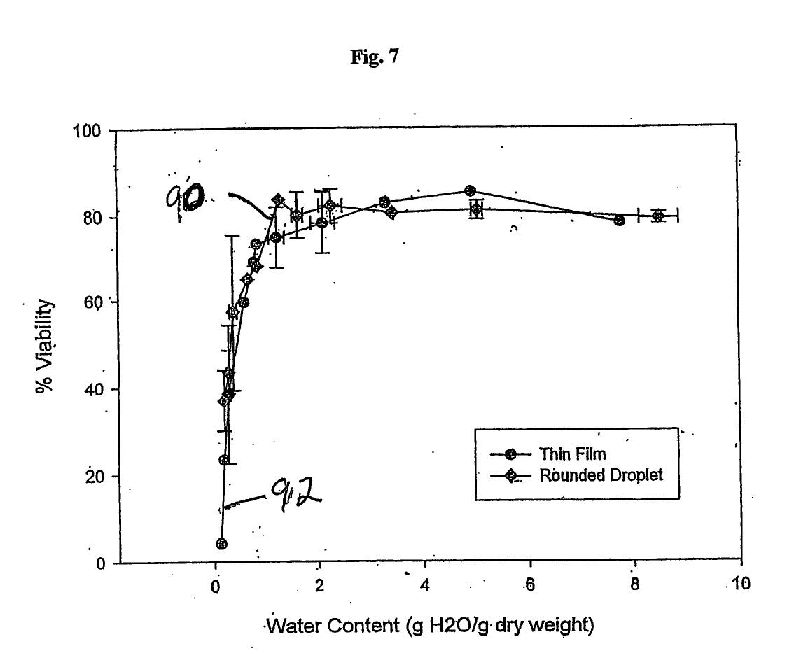

In FIG. 6 there is seen a graph of survival (% viability) vs. water content (gm. water/gm. dry weight) for a first batch of transfected 293 cells (T-293 cells) after air drying while in a thin film configuration and after rehydration, and for a second batch of transfected 293 cells (T-293 cells) after air drying while in a plurality of droplets configuration and after rehydration, with both batches of the transfected 293 cells having trehalose internally. In FIG. 7 there is seen a graph of survival (% viability) vs. water content (gm. water/gm. dry weight) for a first batch of transfected 293 cells (T-293 cells) after vacuum drying while in a thin film configuration and after rehydration, and for a second batch of transfected 293 cells (T-293 cells) after vacuum drying while in a plurality droplet configuration and after rehydration, with both batches of the transfected 293 cells having trehalose internally.

A thin film configuration for the drying solution or buffer has a film containing cells and has a thickness from about 0.1 mm to about 8.0 mm, preferably from about 0.50 mm to about 3.00 mm. A droplet or bead configuration for the drying solution or buffer contains cells and has a bead or droplet physical configuration. When the loaded transfected 293 cells (T-293 cells) are to be dried in a drying solution having rounded droplets or beads, each droplet or bead would have an average volume ranging from about 10 μL to about 250 μL, preferably from about 20 μL to about 150 μL, more preferably from about 30 μL to about 100 μL, most preferably from about 40μ to about 70 μL (e.g., about 50 μL).

Example VII below provides the more specific testing conditions and parameters which produced the graphical illustrations of FIG. 6 and of FIG. 7. Graph 60 in FIG. 6 illustrates the viability (% viability) following rehydration of air-dried, rounded (i.e., bead-shaped) droplets of drying solution containing trehalose-loaded transfected 293 cells. Graph 62 in FIG. 6 illustrates the viability (% viability) following rehydration of an air-dried, thin film drying solution containing trehalose-loaded transfected 293 cells. Graph 90 in FIG. 7 illustrates the viability (% viability) following rehydration of vacuum-dried, rounded (i.e., bead-shaped) droplets of drying solution containing trehalose-loaded transfected 293 cells. Graph 92 in FIG. 7 illustrates the viability (% viability) following rehydration of a vacuum-dried, thin film drying solution containing trehalose-loaded transfected 293 cells.

FIG. 6 broadly illustrates that cell survival increases (e.g., increases by from about 20% to about 90%) by air drying rounded (i.e., bead-shaped) droplets of drying solution containing trehalose-loaded transfected 293 cells to a water content of less than or equal to about 3 grams of water per gram of dry weight of T-293 cells, instead of air drying in thin film configuration the drying solution containing trehalose-loaded transfected 293. FIG. 6 also broadly illustrates that when the drying solution containing trehalose-loaded, p26 transfected T-293 cells are air dried in a thin film configuration (instead of or as opposed to a rounded droplet configuration) to the extent that the trehalose-loaded, p26 transfected T-293 cells had a residual water content of greater than (or equal to) about 3.0 grams of water per gram of dry weight of T-293 cells, survival (% viability) of the 293 cells increases after rehydration. Thus, when air drying is the drying technique for trehalose-loaded, p26 transfected T-293 cells, survival (% viability) is greatest if the drying solution containing the trehalose-loaded, p26 transfected T-293 cells is in thin film configuration and if the residual water content of the trehalose-loaded, p26 transfected T-293 cells is maintained at greater than (or equal to) about 3.0 grams of water per gram of dry weight of T-293 cells (e.g., from about 3.0 grams of water per gram of dry weight of T-293 cells to about 8.0 grams water per gram of dry weight of T-293 cells); and rounded droplets configuration is the preferred configuration for the drying solution containing trehalose-loaded, p26 transfected T-293 cells if the residual water content of the trehalose-loaded, p26 transfected T-293 cells is maintained at less than (or equal to) about 3.0 grams water per gram of dry weight of T-293 cells. As shown in FIG. 5, the survival of the 293 cells (i.e., the biological sample) is preferably at least about 600 (e.g., such as from about 60% to about 80%), more preferably at least about 80%.

FIG. 7 broadly illustrates that cell survival increases (e.g., increases by from about 5% to about 20%) by vacuum drying rounded (i.e., bead-shaped) droplets of drying solution containing trehalose-loaded transfected 293 cells to a water content ranging from a value greater than or equal to about 1 grams of water per gram of dry weight of T-293 cells to a value less than or equal to about 3.5 grams of water per gram of dry weight of T-293 cells, instead of vacuum drying in thin film configuration the drying solution containing trehalose-loaded transfected 293. FIG. 7 also broadly illustrates that when the drying solution containing trehalose-loaded, p26 transfected T-293 cells are vacuum dried in a thin film configuration (instead of or as opposed to a rounded droplet configuration) to the extent that the trehalose-loaded, p26 transfected T-293 cells had a residual water content ranging from a value greater than (or equal to) about 3.5 grams of water per gram of dry weight of T-293 cells to a value less than or equal to about 7.5 grams of water per gram of dry weight of T-293 cells, survival (% viability) of the 293 cells increases after rehydration. Thus, when rounded droplets are to be configuration for drying the drying solution containing trehalose-loaded, p26 transfected T-293 cells, drying may be by either air drying or vacuum drying if the residual water content of the trehalose-loaded, p26 transfected T-293 cells is maintained at less than (or equal to) about 3.0 grams of water per gram of dry weight of T-293 cells; and a thin film configuration is the preferred configuration for the drying solution containing trehalose-loaded, p26 transfected T-293 cells if the residual water content of the trehalose-loaded, p26 transfected T-293 cells is maintained at greater than (or equal to) about 3.0-grams water per gram of dry weight of T-293 cells.

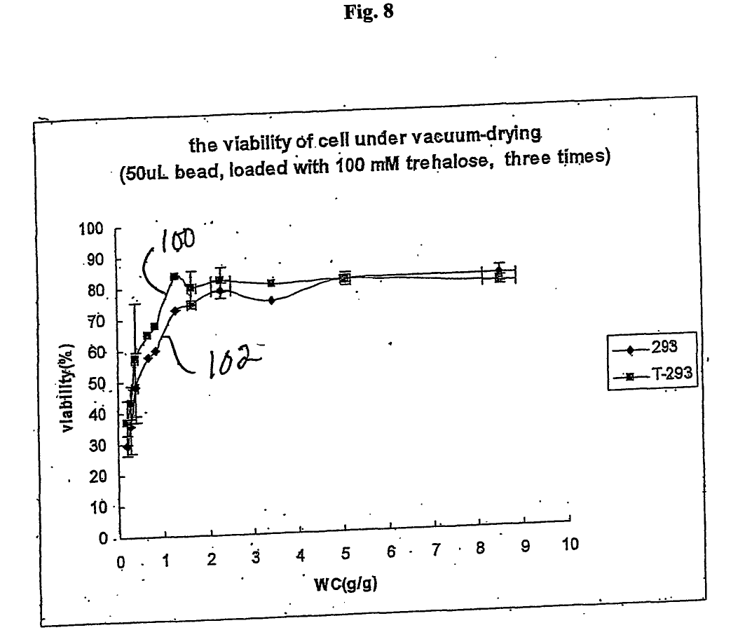

In FIG. 8 there is a graph of survival (% average viability) vs. water content (gm. water/gm. dry weight) for 293 cells (293 cells) after vacuum drying while in a plurality droplet configuration and after rehydration, and for transfected 293 cells (T-293 cells) after vacuum drying while in a plurality droplet configuration and after rehydration, with both the 293 cells and the transfected 293 cells having trehalose internally (e.g., from about 25 mM to about 800 mM of internal trehalose). Example VIII below provides the more specific testing conditions and parameters which produced the graphical illustrations of FIG. 8. Graph 100 in FIG. 8 illustrates the viability (% average viability) following rehydration of vacuum-dried, rounded (i.e., bead-shaped) droplets of drying solution containing trehalose-loaded transfected 293 cells. Graph 102 in FIG. 8 illustrates the viability (% average viability) following rehydration of a vacuum-dried, rounded (i.e., bead-shaped) droplets of drying solution containing trehalose-loaded 293 cells (i.e., trehalose-loaded non-transfected 293 cells). FIG. 8 broadly illustrates that cell survival increases (e.g., increases by from about 10% to about 20%) by vacuum drying rounded (i.e., bead-shaped) droplets of drying solution containing trehalose-loaded transfected 293 cell's to a water content of less than or equal to about 5 grams of water per gram of dry weight of T-293 cells (e.g., from about 0.1 grams of water per gram of dry weight of T-293 cells to about 5.0 grams water per gram of dry weight of T-293 cells), instead of vacuum drying rounded (i.e., bead-shaped) droplets of drying solution containing trehalose-loaded 293 cells (i.e., trehalose-loaded non-transfected 293 cells).

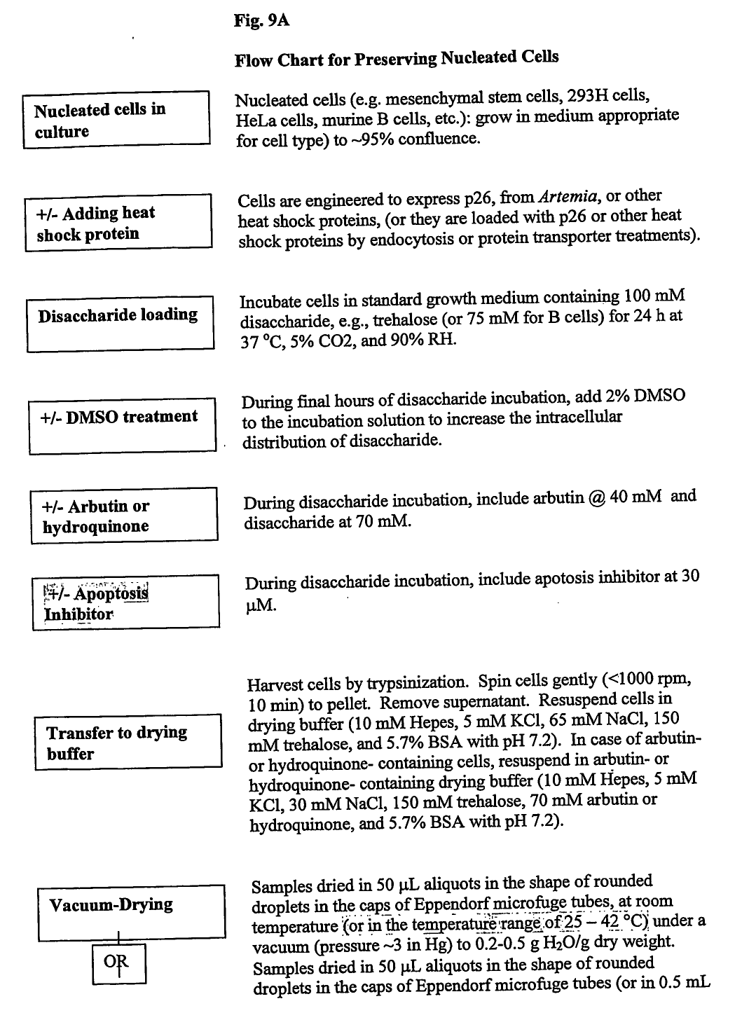

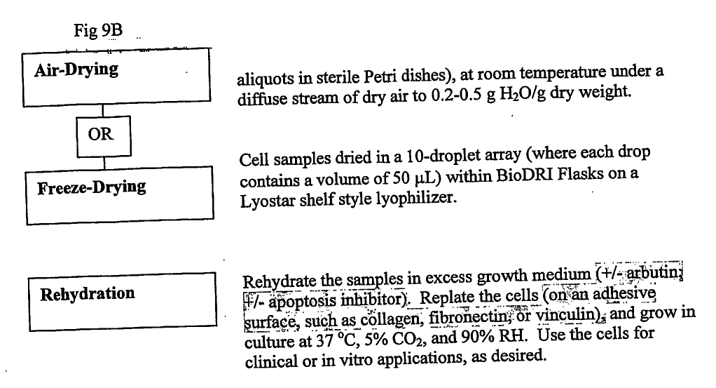

FIG. 9 sets forth a flow chart of a preferred embodiments of the invention.

Embodiments of the present invention will be illustrated by the following set forth examples which are being given to set forth by way of illustration only and not by way of limitation. It is to be understood that all materials, chemical compositions and procedures referred to below, but not explained, are known to those artisans possessing skill in the art. All materials and chemical compositions whose source(s) are not stated below are readily available from commercial suppliers, which are also known to those artisans possessing skill in the art. Parameters such as concentrations, mixing proportions, temperatures, rates, compounds, etc., set forth in these examples are not to be construed to unduly limit the scope of the invention. Abbreviations used in the examples, and elsewhere, are as follows:

DMSO=dimethylsulfoxide; ADP=adenosine diphosphate

PGE1=prostaglandin E1; HES=hydroxy ethyl starch

FTIR=Fourier transform infrared spectroscopy

EGTA=ethylene glycol-bis(2-aminoethyl ether) N,N,N′,N′, tetra-acetic acid

TES=N-tris (hydroxymethyl) methyl-2-aminoethane-sulfonic acid

HEPES=N-(2-hydroxyl ethyl) piperarine-N′-(2-ethanesulfonic acid)

PBS=phosphate buffered saline; HSA=human serum albumin

BSA=bovine serum albumin; ACD=citric acid, citrate, and dextrose

MPCD=methyl-β-cyclodextrin

### EXAMPLE I

p26 was purified from encysted embryos of A franciscana (San Francisco Bay) purchased from San Francisco Bay Brand, Newark, Calif., USA. Purification steps were performed at 4° C. or on ice. Dried embryos (50 g), were hydrated at 4° C. for 16 hours in sea water; filtered; washed with cold 40 mM HEPES-KOH, pH 7.5, at 4° C., 70 mM NaCl, and 1 mM EDTA (buffer A); and homogenized in the same buffer with a Retsch motorized mortar and pestle (Brinkman Instruments, Canada). The homogenate was centrifuged (4000×g, 20 minutes) and the supernatant filtered through 6 layers of cheesecloth, centrifuged again at 16 000×g for 40 minutes, and then at 23 500×g for 30 minutes. Solid (NH₄)₂SO₄ was added to 40% saturation in the final supernatant. Precipitated proteins were collected at 10 000×g for 30 minutes; dissolved in 20 mM Tris-HCl, pH 8.15, 150 mM NaCl, 1 mM MgCl₂, and 0.1 mM EDTA (buffer B); and dialyzed overnight against this buffer. After dialysis, the solution was passed through a 0.45-mm filter, applied to a Source 15 Q ion-exchange column (Amersham Pharmacia Biotech), equilibrated, and developed at 2 mL/min in buffer B. The column was washed with buffer B for 30 minutes, and a linear NaCl gradient (150-500 mM) was used for elution of p26 between 235-270 mM NaCl. Fractions containing p26 were pooled; concentrated using Centriprep-30 (Amicon); dialyzed against 40 mM HEPES-KOH, pH 7.5 (buffer C), and 3.00 mM NaCl; further purified by gel filtration using a TSK-Gel G4000SW_{XL} column (0.78×30 cm, Toso Haas, Japan); equilibrated; and developed at 0.5 mL/min in buffer C and 300 mM NaCl. p26 was eluted between 9.5-10.5 mL of the buffer volume, and the resulting protein was more than 95% pure. The protein was concentrated to approximately 1 mg/mL with Centriprep-30; dialyzed against 50 mM Tris-HCl, pH 8, and 2 mM EDTA (TE buffer); and centrifuged at 10 000×g for 15 minutes. Further concentration and storage in buffer C led to unwanted insoluble aggregates. Aliquots were quick frozen in liquid nitrogen and stored at −70° C. Fractions from each step of purification were checked by sodium dodecyl sulfate-polyacrylamide gel electrophoresis and/or Western immunoblotting with polyclonal antibody to p26 and then pooled according to purity.

### EXAMPLE II

293 cells and T293 cells were grown in T-25 flasks to ˜90% confluence. The cells were harvested by trypsinization according to standard protocols. Briefly, the medium was removed from the cultures and they were washed one time with 5 mL DPBS. Trypsin (1 mL of 0.05% in 0.53 mM EDTA-4Na) was added to the culture for ˜1 min and the flasks were rapped to dislodge the cells. Medium (4 mL) was added to stop the reaction, and the cells were pelleted by centrifugation at 176×g for 5 min. The pellet was suspended in 5-10 mL DPBS and the centrifugation step was repeated. The cell pellet was then suspended in air drying buffer lacking trehalose (10 mM Hepes, 5 mM KCl, 65 mM NaCl, and 5.7% BSA with pH 7.2) at 1.4 million cells per mL. Aliquots (1.0 mL) were placed in 35 mm polysterene Petri dishes and air-dried in a ThermoForma biosafety cabinet in specific marked locations in the center of the hood over 0-24 hours. At various time points during drying, samples were removed for viability and water content analyses. Water contents were measured gravimetrically in triplicate. For viability measurements, samples were rehydrated with 1 mL medium. 50 μL of cellular suspension was mixed with 50 μL trypan blue and incubated at room temperature for 3 min. Cells were visualized at 10× by light microscopy on a hemacytometer and counted using five counts of 50-100 cells per 1 mm² hemocytometer grid square for each sample. Percent viability was calculated as the number of cells excluding the dye divided by the total number of cells. Viability was plotted as a function of water content as the means+/−standard deviation (for both variables), with the results illustrated in FIG. 1.

### EXAMPLE III

293 cells and T293 cells were grown in T-25 flasks to ˜90% confluence. The cells were harvested by trypsinization according to standard protocols. Briefly, the medium was removed from the cultures and they were washed one time with 5 mL DPBS. Trypsin (1 mL of 0.05% in 0.53 mM EDTA-4Na) was added to the culture for ˜1 min and the flasks were rapped to dislodge the cells. Medium (4 mL) was added to stop the reaction, and the cells were pelleted by centrifugation at 176×g for 5 min. The pellet was suspended in 5-10 mL DPBS and the centrifugation step was repeated. The cell pellet was then suspended in air drying buffer containing trehalose (10 mM Hepes, 5 mM KCl, 65 mM NaCl, 150 mM Trehalose, and 5.7% BSA with pH 7.2) at 1.4 million cells per mL. Aliquots (1.0 mL) were placed in 35 mm polysterene Petri dishes and air-dried in a ThermoForma biosafety cabinet in specific marked locations in the center of the hood over 0-24 hours. At various time points during drying, samples were removed for viability and water content analyses. Water contents were measured gravimetrically in triplicate. For viability measurements, samples were rehydrated with 1 mL medium. 50 μL of cellular suspension was mixed with 50 μL trypan blue and incubated at room temperature for 3 min. Cells were visualized at 10× by light microscopy on a hemacytometer and counted using five counts of 50-100 cells per 1 mm² hemocytometer grid square for each sample. Percent viability was calculated as the number of cells excluding the dye divided by the total number of cells. Viability was plotted as a function of water content as the means+/−standard deviation (for both variables), the results shown in FIG. 2.

### EXAMPLE IV

293 cells and T293 cells were grown in T-25 flasks to ˜90% confluence. The cells were incubated in medium with 100 mM trehalose for 24 hours at 37° C. to induce endocytotic loading. The cells were then harvested by trypsinization according to standard protocols. Briefly, the medium was removed from the cultures and they were washed one time with 5 mL DPBS. Trypsin (1 mL of 0.05% in 0.53 mM EDTA-4Na) was added to the culture for ˜1 min and the flasks were rapped to dislodge the cells. Medium (4 mL) was added to stop the reaction, and the cells were pelleted by centrifugation at 176×g for 5 min. The pellet was suspended in 5-10 mL, DPBS and the centrifugation step was repeated. The cell pellet was then suspended in air drying buffer containing trehalose (10 mM Hepes, 5 mM KCl, 65 mM NaCl, 150 mM Trehalose, and 5.7% BSA with pH 7.2) at 1.4 million cells per mL. Aliquots (1.0 mL) were placed in 35 mm polysterene Petri dishes and air-dried in a ThermoForma biosafety cabinet in specific marked locations in the center of the hood over 0-24 hours. At various time points during drying, samples were removed for viability and water content analyses. Water contents were measured gravimetrically in triplicate. For viability measurements, samples were rehydrated with 1 mL medium. 50 μL of cellular suspension was mixed with 50 μL trypan blue and incubated at room temperature for 3 min. Cells were visualized at 10× by light microscopy on a hemacytometer and counted using five counts of 50-100 cells per 1 mm² hemocytometer grid square for each sample. Percent viability was calculated as the number of cells excluding the dye divided by the total number of cells. Viability was plotted as a function of water content as the means+/−standard deviation (for both variables), with the results shown in FIG. 3.

### EXAMPLE V

293 cells and T293 cells were grown in T-25 flasks to ˜90% confluence. The cells were incubated in medium with 100 mM trehalose for 24 hours at 37° C. to induce endocytotic loading. The cells were then harvested by trypsinization according to standard protocols. Briefly, the medium was removed from the cultures and they were washed one time with 5 mL DPBS. Trypsin (1 mL of 0.05% in 0.53 mM EDTA-4Na) was added to the culture for ˜1 min and the flasks were rapped to dislodge the cells. Medium (4 mL) was added to stop the reaction, hand the cells were pelleted by centrifugation at 176×g for 5 min. The pellet was suspended in 5-10 mL DPBS and the centrifugation step was repeated. The cell pellet was then suspended in air drying buffer containing trehalose(10 mM Hepes, 5 mM KCl, 65 mM NaCl, 150 mM Trehalose, and 5.7% BSA with pH 7.2) at 1.4 million cells per mL. Aliquots (1.0 mL) were placed in 35 mm polysterene Petri dishes and air-dried in a ThermoForma biosafety cabinet in specific marked locations in the center of the hood. Samples were then removed and rehydrated with 1 mL medium. Following viability testing by trypan blue exclusion (which used 50 mL), the remaining 950 mL was combined with 7 mL medium, and replated in a T-25 flask. Parallel samples were assayed for residual water content by gravimetric analysis. The cultures were incubated at 37° C., 90% relative humidity, and 5% CO₂ for 24 hours, after which time the medium was removed and replaced with fresh medium. After incubation for 6 more days under the same conditions, the medium was removed and the colonies were stained with Hema 3 and counted. The results are illustrated in FIG. 4, which is a graph of the number of colonies formed after rehydration vs water content after drying the transfected 293 cells (T-293 cells) and the control 293 cells (293-cells) to 0.3 gm. water/gm. dry weight and to 0.2 gm. water/gm. dry weight, with both the transfected 293 cells and the control 293 cells having trehalose internally and with the drying buffer for both the transfected 293 cells and the control 293 cells having 150 mM trehalose.

### EXAMPLE VI

293 cells transfected to produce the protein P26 from Artemia were loaded with trehalose for 24 hours by incubation at 37° C. in medium+100 mM trehalose, which resulted in internally trehalose concentration in the range 20-40 mM. The cells were dried by either air-drying or vacuum-drying and the viability following rehydration was compared by trypan blue exclusion. The air-dried samples (50 μL) were placed at room temperature (˜20° C.) in a modified desiccator flushed with dry air at approximately 200 mL/min. The vacuum-dried samples (50 pL) were placed in a vacuum chamber at room temperature and subjected to a vacuum of approximately 23 inches Hg. The residual water contents were measured by gravimetric analysis. The vacuum-dried samples show a significantly 1 left-shifted curve as compared to the air-dried samples, indicating a much higher viability at the lowest water contents.

### EXAMPLE VII

Transfected 293 cells were dried in different physical configurations to determine the effect of the physical structure of the sample on viability following rehydration. 293 cells transfected to produce the protein P26 from Artemia were loaded with trehalose for 24 hours by incubation at 37° C. in medium+100 mM trehalose, which results in an internal trehalose concentration in the range of 20-40 mM. The cells were dried by either air-drying or vacuum-drying and the viability following rehydration was compared by trypan blue exclusion. The air dried samples (50 μL) were placed at room temperature (˜20° C.) in a modified desiccator flushed with dry air at approximately 200 mL/min. The vacuum-dried samples (50 μL) were placed in a vacuum chamber at room temperature and subjected to a vacuum of approximately 3 inches Hg. The residual water contents were measured by gravimetric analysis. Cells were air-dried or vacuum-dried from either a 50 μL thin film or a 50 μL rounded droplet. Above 2 gH₂O/g dry weight, there is little effect of the physical structure. But, at the lowest water contents, the viabilities are 20-40% higher when the samples are dried in a rounded droplet instead of a thin film.

### EXAMPLE VIII

Average viabilities (+/−SD) of vacuum-dried 293 cells and T293 cells when dried as rounded 50 μL droplets (beads). 293 cells transfected to produce the protein p26 from Artemia and control 293 cells were loaded with trehalose for 24 hours by incubation at 37° C. in medium+100 mM trehalose, which results in an internal trehalose concentration in the range of 20-40 mM. The cells were dried by vacuum-drying and the viability following rehydration was compared by trypan blue exclusion. The vacuum-dried samples (50 μL) were placed in a vacuum chamber at room temperature and subjected to a vacuum of approximately 3 inches Hg. The residual water contents were measured by gravimetric analysis. Although both types of cells show improved viability with this combination, as compared to air-dried samples, the transfected cells still show higher survival than the standard 293 cells at water contents below 2gH₂O/g dry weight. Using this combination of treatments, the viability for the transfected cells is approaching 40% at 0.2gH₂O/g dry weight. This is a significant improvement over methods described in previous disclosures.

### CONCLUSION

Embodiments of the present invention provide that mammalian cells (e.g., T-293 cells) transfected with the stress protein p26 and loaded with trehalose, a sugar found at high concentrations in organisms that normally survive dehydration, survived drying at water contents of about 0.5 gm water/gm dry weight cells, and below about 0.5 gm water/gm dry weight cells. Drying of the cells may be in any suitable manner, such as air drying or vacuum drying. Control mammalian cells not transfected with the stress protein p26, by contrast to the transfected mammalian cells, have diminished survival at water contents as high as 2 gm water/gm dry weight cells. Thus, transfection of mammalian cells with p26 and loading with trehalose improves the ability to dry mammalian cells, particularly mammalian nucleated cells.

### EXAMPLE IX

This Example sets forth a procedure for conducting an exemplar assay for ascertaining entry of a solute into a cell.

Loading of Lucifer Yellow CH into Cells. A fluorescent dye, lucifer yellow CH (LYCH), can be used as a marker for penetration of cell membranes by a solute. Washed cells are incubated in the presence of 1-20 mg/ml LYCH. Incubation temperatures and incubation times can be chosen as desired. After incubation, the cells are spun at 20× at 14,000 RPM on a table centrifuge, resuspended in buffer, spun down for 20 s in buffer and resuspended. Cell counts are obtained on a suitable counter, such as a hemacytometer, and the samples pelleted (for example, by centrifugation for 45 s 25 at 14,000 RPM, table centrifuge). The pellet is lysed in 0.1% Triton buffer (10 mM TES, 50 mM KCl, pH 6.8). The fluorescence of the lysate is measured on a Perkin-Elmer LSS spectrofluorimeter with excitation at 428 nm (SW 10 nm) and emission at 530 run (SW 10 nm). Uptake is calculated for each sample as nanograms of LYCH per cell using a standard curve of LYCH in lysate buffer.

Visualization of cell-associated Lucifer Yellow. LYCH loaded platelets can be viewed on a fluorescence microscope (Zeiss) employing a fluorescein filter set for fluorescence microscopy. Cells can be studied either directly after incubation or after fixation with 1% paraformaldehyde in buffer. Fixed cells can be settled on poly-L-lysine coated cover slides and mounted in glycerol.

Quantification of Trehalose and LYCH Concentration. Uptake is calculated for each sample as micrograms of trehalose or LYCH per cell. The internal trehalose concentration can be calculated assuming a standard cell radius and by assuming that 50% of the cell volume is taken up by the cytosol (rest is membranes). The loading efficiency was determined from the cytosolic trehalose or LYCH concentration and the concentration in the loading buffer.

### EXAMPLE X

This Example sets forth materials and methods for studies of the effect of arbutin in the drying and rehydration of MSCs.

Materials and Methods

Materials

Tissue culture reagents were from Invitrogen (Carlsbad, Calif.), unless otherwise stated. Tissue culture disposables were from Nalge Nunc International (Rochester, N.Y.). Trehalose was from Cargill. (Minneapolis, Minn.). Equipment for western blots, and reagents were from Bio-Rad (Hercules, Calif.) unless otherwise stated. Bovine serum albumin (BSA), and glycine was from Research Organics (Cleveland, Ohio), Bromodeoxyuridine, and anti-bromodeoxyuridine, (mouse IgGₗ, monoclonal PRB-1, Alexa Fluor® 488 conjugate) was obtained from Molecular Probes (Eugene, Oreg.). Arbutin, ascorbic acid, silver nitrate, propidium iodide, and dexamethasone were from Sigma-Aldrich (St Louis, Mo.) and β-glycerophosphate was from Calbiochem (San Diego, Calif.).

Cell Culture

Human MSCs previously isolated from bone marrow and expanded in vitro to passage number 1 were a gift from Osiris Therapeutics (Baltimore, Md.) and were shipped to UC Davis in liquid nitrogen. The cells were grown in Dulbecco's modified Eagle medium-low glucose, with 10% FBS (Hyclone, Logan, Utah) at 37° C. with 5% CO₂ and 90% RH. The cells were used up through passage number 4 at a level of 90-95% confluence. Cells were harvested by washing once with Dulbecco's PBS (DPBS) and incubating for 5-7 min with trypsin-EDTA [0.05% trypsin, and 0.53 mM EDTA-4Na]. This cell suspension was pelleted at 167×g for 10 min and resuspended in medium or the specified buffer. Unused cells were counted before freezing and were frozen in mixture of 10% DMSO, 5% human serum albumin, and 70% Plasma Lyte A (both from Baxter Healthcare Corp., Deerfield, Ill.) until they were needed.

Solute Loading

Cells were grown in T-75 flasks to 90-95% confluence and loaded with extracellular solutes. Briefly, medium was removed from the flasks and replaced with MSC growth medium containing 100 mM trehalose or 70 mM trehalose plus 40 mM arbutin for 24 hours at 37° C. Following incubation, the cells were washed once with 10 mL DPBS and harvested by trypsinization, as described above. The MSCs were then transferred to one of three different drying buffers. The control (no trehalose) drying buffer contained 10 mM Hepes, 5 mM KCl, 140 mM NaCl, with pH 7.2. The trehalose-only drying buffer contained 10 mM Hepes, 5 mM KCl, 65 mM NaCl, 150 mM trehalose, and 5.7% BSA with pH 7.2, and the trehalose-plus-arbutin drying buffer included 10 mM Hepes, 5 mM KCl, 30 mM NaCl, 150 mM trehalose, 70 mM arbutin, and 5.7% BSA with pH 7.2.

Vacuum-Drying

The samples were dried in 50 μL aliquots in the shape of rounded droplets in the caps of Eppendorf microfuge tubes (Online Products, Petaluma, Calif.), at room temperature under a vacuum (pressure ˜3 in Hg). At various time points, parallel samples were removed for assessment of viability and residual water content.

Viability was measured by propidium iodide (PI) exclusion as follows. Rehydrated MSCs were incubated in 2 μg/ml PI for 10 min, then loaded on a hemacytometer (Hausser Scientific, Horsham, Pa.) and examined using an Olympus BX 61 fluorescence microscope (Miami, Fla.). The total number of cells was counted by differential interference contrast microscopy, and the number of dead cells was counted by fluorescence using the Tritc/Di (U-N41002a) from Chroma Technology Corporation (Rockingham, Vt.). Five counts of 50-100 cells per 1 mm² hemocytometer grid square were taken for each sample.

Gravimetric analysis was used to measure the residual water content of the vacuum-dried samples. Samples were weighed on a R 180 D, model Sartorius balance (Westbury, N.Y.) immediately following removal from the vacuum chamber (=vacuum-dried sample) and again following complete water removal by incubation at 60° C. under vacuum (pressure˜3 in Hg) for 24-48 h until a constant mass was achieved (=anhydrous sample). The difference between the anhydrous weight (includes vessel) and the vessel tare was taken as the dry weight of the sample. The difference between the weight of the vacuum-dried sample and that of the anhydrous sample (both include vessel) was taken as the water weight. The residual water contents are reported as the g water per g dry weight of the sample (g H₂O/g dry weight).

Rehydration and Cellular Recovery

Vacuum-dried samples were rehydrated with excess medium (150 uL per sample) and mixed by gentle pipetting. The rehydrated cells were then replated in Lab-Tek slides and incubated overnight. They were then incubated in medium containing 10% alamarBlue (Bio Source, Camarillo, Calif.) for 24 hours. AlamarBlue is reduced by actively metabolizing cells, and only the reduced from is fluorescent. Thus, cellular metabolism can be monitored by alamarBlue fluorescence (REF). Aliquots of medium (1.0 mL) were measured on a Perkin Elmer LS50B luminescence spectrometer (Ex 530 nm, Em 585 nm).

Cell division was monitored by incorporation of bromodeoxyuridine (BrdU). Rehydrated cells were replated as described above and cultured for three weeks in order to gain enough cells to acquire accurate cell counts. The cells were then re-plated at a 1:3 split and pulsed for 2 d with 10 μM BrdU. The samples were washed twice with 1 mL DPBS, and fixed by incubation with 1:1 of DPBS: methanol overnight at 4° C. temperature. The cells were permeabilized with 0.01% Tritron X 100 solution in DPBS for 5 minutes, washed and incubated with 2N HCl for 30 minutes at 37° C. They were then blocked with 1% BSA for 45 minutes at 37° C. and were stained with fluorescently tagged antibodies to the BrdU (Alexa 488) (1:20 dilution) for 45 minutes at 37° C. and propidium iodide (2 μg/mL PI for 10 min). Using an Olympus BX 61 fluorescence microscope, the total cellular population was visualized by the propidium iodide (red) staining, and dividing cells were visualized with the Alexa 488 (green) staining using appropriate TRITC and FITC channels, respectively. Statistical analysis for this and other experiments was conducted using a one-way analysis of variance (ANOVA) with Sigma-Stat software (Jandel Scientific, San Rafael, Calif.).

Osteogenic Differentiation

MSCs were dried and rehydrated as described above. The cells were then replated in Lab-Tek slides and incubated under normal growth conditions (37° C., 5% CO₂, 90% RH), either in the presence of absence of osteogenic supplements (OS). OS medium consisted of D-MEM supplemented with 10% FBS (v/v), 0.1 μM dexamethasone, 50 μM ascorbic acid-2-phosphate, and 10 mM β-glycerophosphate. The cells were fed every 3-4 days by removing and replacing the medium (+/−osteogenic supplements). The cells were grown for two weeks (+/−OS supplements) before usage in the calcium deposition assay.

Differentiation along the osteogenic lineage was assessed by conducting a von Kossa stain for calcium deposition. Briefly, one well of a 2-well Lab-Tek slide was used for each sample. The medium was removed and all wells were rinsed twice with DPBS, and then fixed with 10% formalin, followed by two additional washes with DPBS. To each well, 1 mL 2% AgNO₃ was added, and the plates were incubated in the dark for 10 min. Following the AgNO₃ incubation, all wells were rinsed three times with water, leaving the last rinse on the cells. The plates were placed on a white background with the lids removed and exposed to bright light for 15 min. Finally, the wells were rinsed again thoroughly with water and air dried in the hood. Observation of a dark brown stain was taken as indication of calcium deposition.

To quantify the calcium deposition, triplicate samples of MSCs were loaded and dried to various water contents with trehalose alone or trehalose plus arbutin, as described above. The samples were rehydrated with excess medium and cultured in the presence of osteogenic supplements for two weeks. Medium was then removed from the samples, which were dissolved with 1 N HCl (1 mL). The calcium present was measured using the calcium quantitation kit from Cima Scientific (Dallas, Tex.), by comparison to a standard curve according to manufacturer's protocols. This assay is based on the principle of o-cresolphthalein binding to calcium which forms a purple complex that can be measured spectrophotometrically at 650 nm.

Western Blot Analysis

MSCs were incubated for 24 h in medium containing 10, 25, 50, or 100 mM arbutin at 37° C., or were not incubated with arbutin (controls). The cells were collected by trypsinization, washed with DPBS, counted on a hemacytometer, and transferred into triple detergent lysis buffer (50 mM Tris-HCl pH 8.0, 150 mM NaCl, 0.02% NaN₃, 0.1% SDS, 5 mM pefablock, 1 μg/mL aprotinin, 1% nonidet P-40, and 0.5% sodium deoxycholate) for 30 min with ˜5 sec vortex intervals every ˜5 min. The suspensions were pelleted on an Eppendorf microfuge at 15,000 rpm for 15 min at 4° C., and the supernatants were recovered. The cell lysates were analyzed for protein content by the Lowry method (BioRad QC protein assay kit), and diluted 1:1 into 2× loading buffer (125 mM Tris-HCl, pH 6.8, 4% SDS, 20% glycerol, 10% beta mercaptoethanol, and bromophenol blue). The proteins were analyzed by SDS PAGE, using a 13% gel and 20 μg protein per lane and transferred onto PDVF membranes in Towbin buffer (25 mM Tris base, 192 mM glycine, 20% methanol, pH 8.3). The blot was cut in half, so staining for HSP70 and HSP27 could be accomplished simultaneously. The blots were blocked with 5% non-fat dry milk, and stained with mouse anti-HSP70 (SPA 810, 1:1000 dilution), or mouse anti-HSP27 (SPA 800, 1:1000 dilution), both from Stressgen Biotechnologies Corporation (Victoria, B.C, Canada), then stained with goat anti-mouse antibody conjugated to alkaline phosphatase, and visualized by incubating with NBT/BCIB (both from Pierce Biotechnology, Inc, Rockford, Ill.). The blots were scanned and quantified using the program Quantity One from Bio-Rad.

### EXAMPLE XI

This Example reports the results of the effects of arbutin on survival of MSCs during drying and rehydration.

Mesenchymal Stem Cells Survive Drying to 0.3 g H₂O/g Dry Weight

MSCs were loaded with trehalose by a 24-h incubation in growth medium containing 100 mM trehalose. The trehalose-loaded cells were harvested by trypsinization and transferred into drying buffer containing trehalose (10 mM Hepes, 5 mM KCl, 65 mM NaCl, 150 mM trehalose, and 5.7% BSA with pH 7.2). In parallel, control cells were harvested by trypsinization, and transferred into drying buffer lacking trehalose (10 mM Hepes, 5 mM KCl, 140 mM NaCl, pH 7.2). Aliquots of cell suspension (50 μL at 1.25×10⁶ cells/mL) were dried in the caps of Eppendorf microfuge tubes by exposure to vacuum (pressure ˜6 in Hg) for a period of 0-5 h. Viability decreased with residual water content. The protective role of trehalose was clear from the data, especially below 2.0 g H₂O/g dry weight, thus trehalose was included in all the subsequent experiments. It was found that ˜50% of the cells survived drying to ˜0.3 g H₂O/g dry weight. That is, to our knowledge, the highest viability reported for nucleated cells taken to this level of dehydration.

Arbutin was also tested as a possible protectant for the MSCs during drying. MSCs were loaded with the protective solutes by a 24-h incubation in growth medium containing 100 mM trehalose or 70 mM trehalose and 40 mM arbutin at 37° C., 5% CO₂, and 90% RH. The samples were then harvested by trypsinization and transferred into drying buffer containing trehalose only (10 mM Hepes, 5 mM KCl, 65 mM NaCl, 150 mM trehalose, and 5.7% BSA with pH 7.2) or trehalose plus arbutin (10 mM Hepes, 5 mM KCl, 30 mM NaCl, 150 mM trehalose, 70 mM arbutin, and 5.7% BSA with pH 7.2). The samples were vacuum dried to four different water contents spanning a large range for residual water (1.34-0.23 g H₂O/g dry weight) and rehydrated with excess medium. The viabilities were measured by propidium iodide (PI) exclusion.

Arbutin provided neither a benefit nor liability to the samples immediately following rehydration, as the trehalose-only and trehalose-plus-arbutin samples showed no significant difference in survival for any water content tested (P=0.174). This result contrasted with the effect of other antioxidants, such as epigallocatechin gallate (EGCG), glutathione, or glutathione ester, which all caused large decreases in viability measured immediately after rehydration. This led us to hypothesize that arbutin might be a valuable protective compound for the MSCs, since there was no drop in immediate viability, and the beneficial effects of arbutin were likely to appear over time in the rehydrated samples.

Arbutin Enhances Recovery of Vacuum-dried MSCs

One method for measuring the recovery of the rehydrated cells is to examine the cellular metabolism of the rehydrated samples. The dye alamarBlue is reduced by actively metabolizing cells, and only the reduced form is fluorescent. MSCs were loaded and vacuum-dried to various water contents with trehalose only or trehalose plus arbutin, as described above. The samples were dried and rehydrated under sterile conditions, and then re-plated in medium containing 10% alamarBlue. After 24 h incubation, the fluorescence of the medium was measured (Ex 530, Em 585) for all samples.

At the highest water contents tested (1.34 and 0.56 g H₂O/g dry weight), there was no difference in the ability of the rehydrated cells to reduce alamarBlue (P=0.588). As the water content was reduced below 0.4 g H₂O/g dry weight, however, a significant difference appeared between the arbutin containing samples and the controls (P=0.030). In fact, at 0.38 g H₂O/g dry weight, there was almost a four-fold increase in the fluorescence of the alamarBlue from the arbutin containing samples, as compared to the trehalose-only samples. In both cases, the fluorescence dropped at the lowest water contents, but in the arbutin containing samples this decrease occured at a much lower water content (0.23 g H₂O/g dry weight) than in the control samples (0.38 g H₂O/g dry weight). This result indicates that at water contents below 0.5 g H₂O/g dry weight, arbutin can provide a significant advantage to cell health over time following rehydration.

Another, more stringent, test for rehydrated MSCs is whether the cells can grow and divide following rehydration. Bromodeoxyuridine (BrdU) can serve to label cells that are actively dividing, as it is only incorporated into newly synthesized DNA. MSCs were vacuum-dried in the presence or absence of arbutin, as described above. The cells were rehydrated under sterile conditions, re-plated, and cultured for 3 weeks, after which they were split 1:3 and pulsed for 2 days with 10 μM BrdU. The cultures were then washed, permeabilized, and stained with fluorescently tagged antibodies to the BrdU (Alexa 488) and propidium iodide. The total cellular population was visualized by the propidium iodide staining, and the dividing cells were visualized with the fluorescent antibodies to BrdU (nuclei stained green).

The arbutin-containing samples had significantly higher cell counts at all except the highest water content tested (P=0.001). This difference was particularly dramatic at the two lowest water contents tested (0.33 and 0.27 g H₂O/g dry weight). In fact, at 0.27 g/g, no cells remained in the trehalose-only samples, but a considerable number of cells were still present in the arbutin containing samples.

Of the cell population present, the line plots show that approximately 70-80% were capable of cell division. There was very little difference between the samples dried in the presence or absence of arbutin for this parameter (P=0.656). The finding that such a large percentage of the rehydrated cells could incorporate BrdU into newly synthesized DNA suggests excellent retention of normal physiological processes, and the larger cell counts found in samples dried in the presence of arbutin provides further evidence that this hydroquinone enables enhanced recovery of dried and rehydrated MSCs.

Arbutin Enhances Osteogenic Differentiation of Vacuum-dried MSCs

We further investigated whether the rehydrated MSCs are capable of differentiation down the osteogenic pathway, as an indication of whether stem cells would retain differentiation capabilities following dehydration and storage. Cells were loaded and vacuum-dried to 0.37 g H₂O/g dry weight in the presence of trehalose or trehalose plus arbutin. The samples were then rehydrated under sterile conditions and cultured for two weeks in the presence or absence of osteogenic supplements (OS, 0.1 μM dexamethasone, 50 μM ascorbic acid-2-phosphate, and 10 mM β-glycerophosphate). Differentiation along the osteogenic lineage was assessed by conducting a von Kossa stain for calcium deposition. Observation of a dark brown stain following treatment with AgNO₃ was taken as indication of calcium deposition.

The samples grown in the absence of osteogenic supplements were negative for von Kossa staining, as expected. Both samples treated with the osteogenic supplements were positive for von Kossa staining, but it was much more pronounced in the samples that were loaded and dried with arbutin and trehalose, in comparison to samples that were loaded and dried with trehalose alone. These results show that the dried and rehydrated cells are indeed competent to differentiate along one of the normal developmental pathways, and that arbutin augments this ability.

Since the difference in the von Kossa staining was quite striking between the samples dried in the presence and absence of arbutin, we quantified the calcium deposition using o-cresolphthalein binding to calcium, which forms a purple complex that can be measured spectrophotometrically. Triplicate samples of MSCs were loaded and dried to various water contents with trehalose alone or trehalose plus arbutin. The samples were rehydrated under sterile conditions and cultured in the presence of osteogenic supplements for two weeks, as described above. The samples were then dissolved with 1 N HCl and the calcium measured using a kit from Cima Scientific and comparison to a standard curve.

The results show that, similar to other measurements of cell health, there was little difference between the samples dried to the highest water contents. In samples dried to 0.47 g H₂O/g dry weight, calcium deposition found in MSC samples dried in the presence or absence of arbutin was virtually identical. In contrast, as water was removed, the difference between the samples became dramatic. At a water content of 0.30 g H₂O/g dry weight, arbutin caused a 25-fold increase in the ability of the cells to deposit calcium. Clearly, arbutin conferred a distinct advantage to the dried MSCs, an advantage which appears over time after rehydration and enables the samples to more effectively differentiate when conditions are appropriate.

Arbutin Induces Expression of HSP70 in MSCs

The chemical structure for arbutin resembles that of aspirin and salicylic acid, two known inducers of the heat shock response. We, therefore, addressed the hypothesis that one mechanism by which arbutin could impart some protective effect under stressful conditions is through inducing the expression of heat shock proteins in the MSCs. A Western blot analysis was conducted for HSP70 and HSP27 on samples incubated for 24 h at 37° C. in the presence of increasing concentrations of arbutin.

Both the blots themselves and a line plot showing the quantitation of the immunostained protein bands indicate that arbutin causes a dose-dependent increase in the expression of HSP70, but not HSP27. The effect on HSP70 at 50 mM arbutin (which is similar to the concentration during the loading phase in the drying experiments), although not as dramatic as that at 100 mM, is still significant (P=0.002 by ANOVA test, SigmaStat software). This result is consistent with a protective effect of arbutin through induction of endogenous heat shock proteins. However, arbutin also has several other beneficial properties, as mentioned above. Thus, it is quite likely that the full protective role of arbutin is the result of a complex series of effects at both the membrane and cellular levels.

### EXAMPLE XII

This Example discusses the results set forth in the previous Example.

The current study has addressed the critical issue of whether dried and rehydrated mesenchymal stem cells can function normally in response to differentiation signals. Two protective compounds were investigated, trehalose and arbutin. Cells dried in the absence of both solutes did not survive well below 2.0 g H₂O/g dry weight. This suggests that earlier reports, in which water contents were not quantified, but that showed high cellular viability or attachment to the growth surface, after drying in the absence of trehalose (e.g. Gordon et al., 2001), were actually investigations of samples containing relatively high water contents.

Samples loaded and dried in the presence of trehalose and arbutin showed little difference in viability from samples loaded and dried with trehalose alone when measured immediately following rehydration. This apparently negative result was actually a promising indication that arbutin could serve as a useful tool in preserving the MSCs during dehydration. Antioxidants can help protect against damaging reactive oxygen species (ROS) generated under mostly dehydrated conditions, but other antioxidants tested, such as EGCG and glutathione, caused large decreases in survival. Thus, the finding that arbutin was not disruptive to membrane integrity immediately after rehydration led to the possibility of exploiting its many protective properties during the various stages of loading, drying, rehydration, and culturing of the MSCs.

Arbutin does not have a universally protective effect, however. In fact, the striking benefit of arbutin to dried and rehydrated MSCs was cell type-specific, as arbutin was toxic to 293H cells. It is well established that the role of arbutin as either stabilizer or destabilizer depends on the lipid composition of the membranes present, and this can help to explain the contrasting effects on different cell types.

Besides arbutin, other amphiphilic compounds are common in desiccation tolerant plant tissues, such as seeds and pollen grains. These compounds quite often partition into membranes to a greater extent during drying than in the fully hydrated state, which is likely to be the case for arbutin as well. The protective role of these compounds remains something of a puzzle, as they often cause membrane leakage when tested in vitro. Their presence in tissues and organisms capable of withstanding dehydration must indicate that their beneficial effects (most are strong antioxidants) outweigh their damaging properties, at least in the region of the specific target membranes where they are found. The current findings indicate that, under specialized conditions, amphiphiles such as arbutin can be used in the preservation of cells or tissues unrelated to those from which they came.

In the days and weeks following rehydration, the samples dried in the presence of arbutin showed much stronger recovery, as measured by cellular metabolism and cell count. In contrast, there was no significant difference between the samples dried in the presence or absence of arbutin with regard to BrdU incorporation. This was very likely a result of experimental protocol, however. In order to have enough cells for an accurate count, the rehydrated cells had to be cultured for three weeks before the BrdU pulsing could take place. During this time, the unhealthy cells could have been lost due to such things as poor attachment. This could have led to the high percentages (70-80%) of the cell populations that were capable of division. Nevertheless, the finding that both treatments produced cell populations capable of cell division, as measured by BrdU incorporation, is a significant advancement in the effort to preserve nucleated cells. Further, arbutin-treated samples did show a strong advantage in relation to the number of cells present after three weeks, especially when the samples were taken to water contents below 0.5 g H₂O/g dry weight. In combination with the results on osteogenic differentiation, these data confirm the ability of arbutin to aid recovery of the dried and rehydrated MSCs.

Based on the similarity of the chemical structure of arbutin with known inducers of the heat shock response, a Western blot analysis for HSP70 and HSP27 was conducted. Indeed, arbutin caused a dose dependent increase in the expression of HSP70. Arbutin has been shown to increase the fluidity of dry and hydrated model membranes, because it inserts with its phenol moiety into the bilayer, and lowers the gel to liquid crystalline phase transition temperature. This is similar to the effect of other fluidizing agents, such as benzyl alcohol, which are known to lower the temperature at which the heat shock response is activated. Thus, arbutin's effect of inducing the expression of HSPs correlates well with the membrane trigger hypothesis for induction of the heat shock response.

It is less likely that arbutin induced the expression of heat shock proteins in the MSCs by causing osmotic stress. Osmotic stress can cause such a response, but the concentrations necessary are much higher. The highest concentration of arbutin used (100 mM) is not sufficient to cause expression of HSPs by this mechanism. Further, 100 mM trehalose had the opposite effect, and actually decreased the expression of heat shock proteins in the MSCs. We therefore suggest that trehalose might lower the level of stress “perceived” by the cells and thus inhibit the heat shock response to a certain degree, a hypothesis we are currently exploring.

The increased expression of heat shock proteins could serve to stabilize proteins and membranes in the MSCs under stressful conditions. In addition, HSP70 has been shown in inhibit apoptosis caused by the leakage of cytochrome C from the mitochondria. Thus, the induction of these proteins could be one main mechanism by which arbutin aids recovery of the MSCs following rehydration.

It is likely that inducing the heat shock response is not the only mechanism by which arbutin affects the cells, however. Arbutin has many varied effects on membranes, including inserting into the lipid bilayer at the phenol moiety, decreasing the phase transition of dry lipid, preventing enzymatic lipid hydrolysis, acting as an anti-oxidant, relaxing negative curvature and stabilizing the lamellar phase of membranes containing non bilayer-forming lipids, and stabilizing certain compositions of membranes to freeze-thaw and drying stresses. Thus, it is reasonable that the added protection that arbutin affords to the MSCs during drying is a complex process involving more than one pathway.

In summary, MSCs loaded and dried in the presence of trehalose showed almost 60% viability after drying to 0.38 g H₂O/g dry weight. Including arbutin in the loading and drying media did not change the viability when measured immediately after rehydration, but dramatically increased recovery of the MSCs, as measured by metabolism and cell count. Both treatments produced rehydrated cells capable of cell division, as measured by BrdU incorporation. When the cells were induced to differentiate down the osteogenic lineage, both treatments resulted in positive von Kossa staining. However, when the cells were dried to the lower water contents, arbutin caused nearly a 25-fold increase in the cells' ability to deposit calcium under osteogenic conditions. The effects of arbutin are likely to result from more than one mechanism, but one possible candidate is the induction of endogenous heat shock proteins, which was shown by Western blot analysis to be a dose-dependent effect of arbutin in MSCs.

### EXAMPLE XIII

This Example shows the transfection of cells with an examplar apoptosis inhibitor and expression of HSPs in cells.

Apoptosis was monitored during dry storage of CANARY cells. CANARY cells are murine B cells designed for use in biosensors. See, Rider et al., Science 301:213-215 (2003). Each line of CANARY cells is a clone specific to detect a certain antigen (e.g. anthrax, plague, smallpox, etc). When the cells detect their specific antigen (or are exposed to an IgM), the internal calcium concentration increases. Because the cells are engineered to express the jellyfish protein aqueorin, when the internal calcium concentration increases, they give off a burst of light that is measurable by a bioluminometer. The presence of the reporter protein makes them convenient to work with; however, the results obtained are expected to be generally applicable.

CANARY cells (4 flasks) were prepared for drying by a 24-h incubation in growth medium, in the presence or absence of 75 mM trehalose, in the presence or absence of 30 μM OPH-109, a pan caspase apoptosis inhibitor (MP Biomedicals), in the presence or absence of 30 μM Caspase 1 inhibitor II and in the presence or absence of 20 μg /ml Bcl -xL (a cytochrome c release inhibitor). The cells were vacuum-dried in 50 μL droplets under the same four conditions at 25° C., to a residual water content of 0.49 g H₂O/g dry weight. Samples were protected by trehalose+OPH-109+Bcl -xL. Viability and apoptosis were quantified using flow cytometry immediately following drying and rehydration, or after 24 or 48 h storage in individually sealed vacuum packets at 4° C. in the dark. The cells were loaded and dried in the presence of both trehalose and OPH-109, which produced nearly 70% viable cells and only ˜25% apoptotic cells following rehydration. We got same results in the presence of trehalose and OPH and Caspase 1 inhibitor II or Bcl-xL, but the effect of Bcl-xL was better than Caspase 1 inhibitor II on the storage process and after storage for 24 h, the viability was still greater than 30%.

We also investigated the bioluminescence after drying CANARY B cells in the presence or absence of 75 mM trehalose, in the presence of both trehalose+30 μM OPH-109, as described above, to a residual water content of 0.71 g H₂O/g dry weight. The bioluminescence was quantified using Sirius Luminometer immediately following drying and rehydration, using IgM as stimulator. The cells that were dried in the absence of trehalose showed a much lower signal in comparison to the undried controls than did the cells loaded and dried in the presence of trehalose or trehalose+OPH. This suggests that trehalose and OPH are very helpful for CANARY B cells during drying

The presence of heat shock proteins in CANARY B cells was investigated by Western blot analysis and immunoostaining. The cells were washed with DPBS, counted on a hemacytometer, and transferred into triple detergent lysis buffer (50 mM Tris-HCl pH 8.0, 150 mM NaCl, 0.02% NaN3, 0.1% SDS, 5 mM pefablock, 1 μg/mL aprotinin, 1% nonidet P-40, and 0.5% sodium deoxycholate) for 30 min with ˜5 sec vortex intervals every ˜5 min. The suspensions were pelleted on an Eppendorf microfuge at 15,000 rpm for 15 min at 4° C., and the supernatants were recovered. The cell lysates were analyzed for protein content by the Lowry method (BioRad QC protein assay kit), and diluted 1:1 into 2× loading buffer (125 mM Tris-HCl, pH 6.8, 4% SDS, 20% glycerol, 10% beta mercaptoethanol, and bromophenol blue). The proteins were analyzed by SDS PAGE, using a 13% gel and 20 μg protein per lane and transferred onto PDVF membranes in Towbin buffer (25 mM Tris base, 192 mM glycine, 20% methanol, pH 8.3). The membranes were blocked with non-fat milk and probed with antibodies to HSP110, HSP90, HSP70, HSP60, HSP27, and α-B-crystallin, as well as secondary antibodies conjugated to alkaline phosphatase. The CANARY B cells strongly expressed HSP110, HSP90, and HSP60.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method for loading a disaccharide into mammalian nucleated cells, comprising:
contacting said cells for at least 2 hours with a solution comprising at least one disaccharide, thereby loading the cells with disaccharide to produce disaccharide-loaded mammalian nucleated cells.

2. A method of claim 1, wherein said cells are selected from the group consisting of stem cells, immune system cells, and epithelial cells.

3. A method of claim 1, wherein said contacting is for 10 hours.

4. A method of claim 1, wherein said contacting is for 24 hours.

5. A method of claim 1, wherein said disaccharide is trehalose.

6. A method of claim 1, wherein said solution further comprises not more than 3% dimethyl sulfoxide.

7. A method for increasing survival of mammalian nucleated cells following drying and rehydration, comprising:
(a) contacting said cells with a solution comprising at least one disaccharide for at least 2 hours, thereby producing disaccharide-loaded cells,
(b) drying said disaccharide-loaded cells to a residual water content between 0.2 and 0.5 gram water per gram of dry weight, and
(c) rehydrating said cells, thereby increasing survival of the cells.

8. A method of claim 7, wherein said contacting is for 24 hours.

9. A method of claim 7, wherein said cells are selected from the group consisting of stem cells, immune system cells, and epithelial cells.

10. A method of claim 7, wherein said disaccharide is trehalose.

11. A method of claim 7, wherein said cells further comprise a heat shock protein.

12. A method of claim 11, wherein said heat shock protein is induced by exposing said cells to a heat shock.

13. A method of claim 12, wherein said heat shock consists of raising the temperature of medium contacting the cells to 42-44° C. for one hour, and then allowing the temperature of the medium to drop to 36-38° C.

14. A method of claim 11, wherein said heat shock protein is introduced into the cells by contacting said cells with a solution comprising said protein.

15. A method of claim 11, wherein said heat shock protein is expressed from a nucleic acid sequence introduced into said cells.

16. A method of claim 11, wherein said heat shock protein is p26 from Artemia franciscana.

17. A method of claim 7, further wherein said cells are contacted with a solution comprising an apoptosis inhibitor.

18. A method of claim 17, wherein said apoptosis inhibitor is selected from the group consisting of N-(2-Quinolyl)valyl-aspartyl-(2,6-difluorophenoxy)methyl ketone (in which the aspartyl residue is o-methylated or non-o-methylated), caspase I inhibitor II, calpain inhibitor, and Bcl-xL.

19. A method of claim 7, further wherein said cells are contacted by a solution comprising arbutin or hydroquinone, provided that said cells are not 293 cells or B cells.

20. A method of claim 7, further wherein said cells are contacted by a solution comprising not more than 3% dimethyl sulfoxide.

21. A method of claim 7, further wherein said cells are contacted by a solution comprising a heat shock protein and an apoptosis inhibitor.

22. A method of claim 21, wherein said solution further comprises not more than 3% dimethyl sulfoxide.

23. A method of claim 19, wherein said cells are dried in a medium comprising arbutin or hydroquinone.

24. A method of claim 7, wherein said cells are dried in rounded droplets of drying buffer.

25. A method for increasing survival of mammalian nucleated cells following drying and rehydration, comprising:
(a) contacting said cells with a solution comprising an apoptosis inhibitor, thereby loading the cells with said apoptosis inhibitor, to produce apoptosis inhibitor-loaded cells,
(b) drying said apoptosis inhibitor-loaded cells, and
(c) rehydrating said cells, thereby increasing survival of the cells.

26. A method of claim 25, wherein said apoptosis inhibitor is selected from the group consisting of N-(2-Quinolyl)valyl-aspartyl-(2,6-difluorophenoxy)methyl ketone (in which the aspartyl residue is o-methylated or non-o-methylated), Caspase I inhibitor II, Calpain inhibitor, and Bcl-xL.

27. A method of claim 25, wherein said cells are selected from the group consisting of stem cells, immune system cells, and epithelial cells

28. A method of claim 25, wherein said cells are dried in droplets of drying buffer.

29. A method for increasing survival of mammalian nucleated cells following drying and rehydration, comprising:
(a) introducing a heat shock protein into, or inducing production of a heat shock protein in, said cells, to produce heat shock protein-loaded cells,
(b) drying said heat shock protein-loaded cells; and
(c) rehydrating said cells, thereby increasing survival of the cells.

30. A method of claim 29, wherein said heat shock protein is p26 from Artemia franciscana.

31. A method of claim 29, wherein said heat shock protein is introduced into said cells by incubating said cells in a medium comprising said heat shock protein.

32. A method of claim 29, wherein said heat shock protein is induced in said cells by raising the temperature of medium contacting the cells to 42-44° C. for one hour, and then allowing the temperature of the medium to lower to 36-38° C.

33. A method of claim 29, wherein said heat shock protein is introduced into said cells by introducing into said cells a nucleic acid sequence comprising a promoter operably linked to a sequence encoding said heat shock protein.

34. A method of claim 29, wherein said cells are selected from the group consisting of stem cells, immune system cells, and epithelial cells.

35. A method of claim 29, wherein said cells are dried in droplets of drying buffer.

36. A method for increasing survival of mammalian nucleated cells following drying and rehydration, provided said cells are not 293 cells or B cells, comprising:
(a) incubating said cells with a compound selected from arbutin and hydroquinone, to produce arbutin- or hydroquinone-loaded cells,
(b) drying said arbutin- or hydroquinone-loaded cells, and
(c) rehydrating said cells, thereby increasing survival of the cells.

37. A method of claim 36, wherein said compound of step (a) is arbutin.

38. An isolated mammalian nucleated cell comprising a disaccharide and a compound selected from the group consisting of arbutin and hydroquinone.

39. An isolated mammalian nucleated cell of claim 38, wherein said compound is arbutin.

40. A mammalian nucleated cell of claim 38, wherein said cell is dried.

41. A mammalian nucleated cell of claim 38, further comprising an apoptosis inhibitor.

42. A mammalian nucleated cell of claim 38, further comprising a heat shock protein.

43. A mammalian nucleated cell of claim 38, wherein said disaccharide is trehalose.

44. An isolated dried mammalian nucleated cell comprising a disaccharide and an exogenous heat shock protein.

45. A dried mammalian nucleated cell of claim 44, wherein said disaccharide is trehalose.

46. A isolated, dried mammalian nucleated cell comprising a disaccharide and an exogenous apoptosis inhibitor.

47. A dried mammalian nucleated cell of claim 46, wherein said disaccharide is trehalose.
